(19) United States
(12) Patent Application Publication
(10) Pub. No.: US 2024/0294457 A1
(43) Pub. Date: Sep. 5, 2024

(54) Strained bi-functionalized trans-cyclooctenes

(71) Applicant: Stichting Radboud Universiteit, Nijmegen (NL)

(72) Inventors: Floris Petrus Johannes Theodorus Rutjes, Wijchen (NL); Luuk Maartense, Nijmegen (NL); Daniel Blanco-Ania, Utrecht (NL)

(73) Assignee: Stichting Radboud Universiteit, Nijmegen (NL)

(21) Appl. No.: 18/571,754

(22) PCT Filed: Jul. 8, 2022
(86) PCT No.: PCT/EP2022/069029

§ 371 Date: Dec. 19, 2023

(30) Foreign Application Priority Data
Jul. 9, 2021 (EP) 21184908.8

(51) Int. Cl. C07C 69/74; A61K 47/54; C07C 35/23; C07C 69/757; C07C 69/96; C07C 271/34; C07D 311/16

(52) U.S. Cl. CPC C07C 69/74; A61K 47/542; C07C 35/23; C07C 69/757; C07C 69/96; C07C 271/34; C07D 311/16; C07C 2602/32

(57) ABSTRACT

The present invention relates to novel bi-functionalized trans-cyclooctenes of formula (1). It also relates to conjugates, compositions, medical uses of such compounds, and methods for producing such compounds as well as conjugates of such compounds.

### FIELD OF THE INVENTION

The present invention relates to novel bi-functionalized trans-cyclooctenes of formula (I). It also relates to conjugates, compositions, medical uses of such compounds, and methods for producing such compounds as well as conjugates of such compounds.

### BACKGROUND ART

Click chemistry such as in vivo click chemistry has been demonstrated as an attractive platform for diagnostic and therapeutic uses, especially when used in antibody-drug conjugates (ADCs). For example, the in vivo click reaction between radiolabelled tetrazines and trans-cyclooctene (TCO) functionalized with an antibody has been shown for this purpose (Fang et al, Tetrahedron. 2019 Aug. 9; 75(32):4307-4317). To enhance the clinical capability of trans-cyclooctenes, however, they would have to comprise multiple handles, and show a fast click reaction, whereby the click reaction induces a release of a releasable moiety. Currently available trans-cyclooctenes do not meet all these criteria.

As a result, there is a need for bi-functionalized trans-cyclooctenes that satisfy these criteria. There is a need for bifunctional TCOs that are synthetically accessible via straightforward routes. There is a need for bifunctional TCOs wherein at least one group releases after a click.

There is a need for bifunctional TCOs that have fast release kinetics after a click.

### SUMMARY OF THE INVENTION

The invention provides a compound of general formula (I), or a salt thereof:

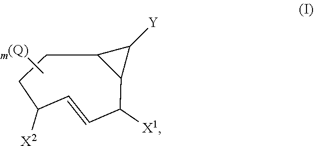

Wherein one of X¹ and X² is —R^{a} or —Z¹—C(═Z²)—R^{b}, and the other is —H; Y is chosen from —C(═Z³)—R^{c}or —CH₂—R^{d}; Z¹, Z², and Z³ are in each instance chosen independently from O, S or NQ^{N}; R^{a}, R^{b}, R^{c}, and R^{d} are in each instance chosen independently from —O—Pr, —S—Pr, —NQ^{N1}Q^{N2}, a leaving group, a a small organic molecule, a pharmaceutically active substance, or a targeting moiety; Pr is in each instance chosen independently from —H, a linear, branched, or cyclic C1-6 alkyl or acyl, or a protecting group, wherein acyl or alkyl are optionally unsaturated and optionally substituted with halogen, alkoxy, —C(═O)O(CH₂)₀₋₄H, or haloalkoxy; Q, Q^{N}, Q^{N1}, and Q^{N2} are in each instance chosen independently from —H or a linear, branched or cyclic C1-6 alkyl, wherein alkyl is optionally unsaturated and optionally substituted with halogen, alkoxy, or haloalkoxy; and m is 0, 1, 2, 3 or 4.

Preferably m is 0. Preferably X² is —R^{a} or —O—C(═O)—R^{b}, and X¹ is —H. Preferably Y is —C(═O)—R^{c} or —CH₂R^{d}. Preferably the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Preferably the leaving group is a halogen, preferably chlorine, bromine, or iodine, more preferably chlorine, or an active ester. In some embodiments the compound is of general formula (V-OO), (V-HO), (V-OH) or (V-HH):

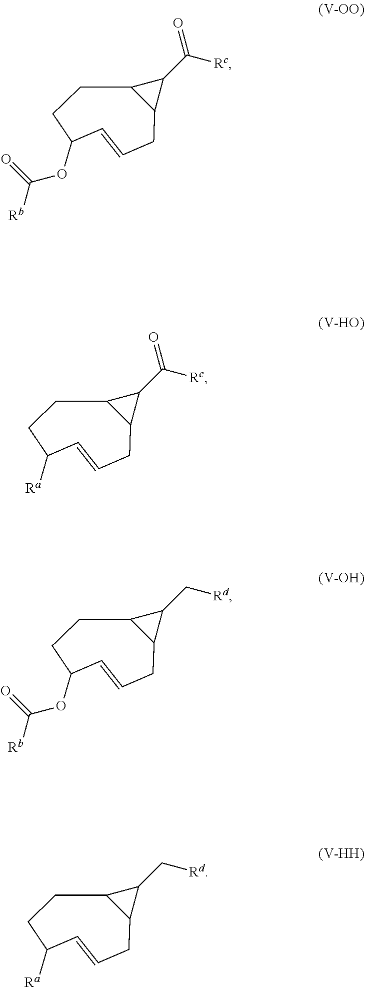

In some embodiments the compound is of general formula (III-a), (III-c), or (III-e):

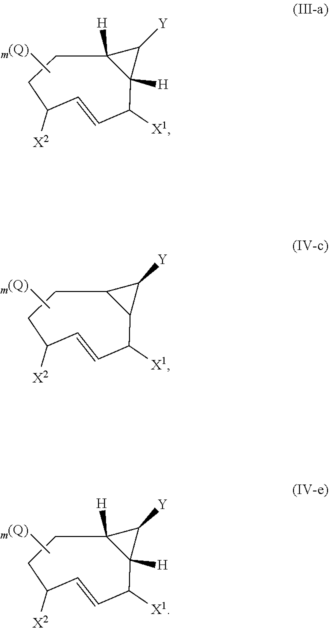

In preferred embodiments the compound is of general formula (II-a) or (VI-a) or an enantiomer thereof:

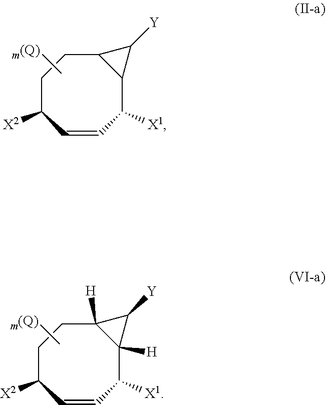

Preferably the compound is of general formula (VII-O) or (VII-OH) or an enantiomer thereof, wherein R^{b}, R^{c} and R^{d} are in each instance chosen independently from —OH or a leaving group:

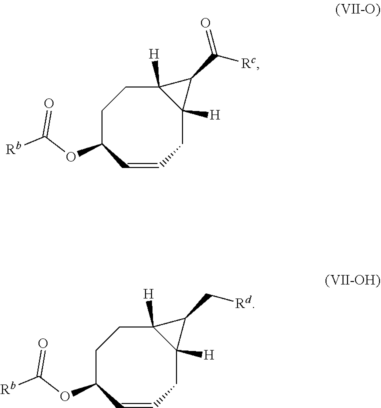

Also provided is a conjugate comprising a moiety conjugated to a compound as defined above, preferably wherein said moiety is a small organic molecule, a pharmaceutically active substance, a macromolecule, or a biomolecule, more preferably wherein said moiety is a protein, most preferably wherein said moiety is an antibody. The invention further provides a method for producing a conjugate as defined above, the method comprising the steps of: i) providing a compound as defined above; ii) providing a second compound, preferably wherein said second compound is a small organic molecule, a pharmaceutically active substance, a macromolecule, or a biomolecule, more preferably wherein said second compound is a protein, most preferably wherein said second compound is an antibody; iii) reacting the compound provided in step i) with the second compound to form a conjugate; and optionally iv) isolating the conjugate obtained in step iii).

The invention further provides a composition comprising a compound as defined above, or a conjugate as defined above, and a pharmaceutically acceptable excipient, preferably wherein the composition comprises a tetrazine, more preferably dipyridyl tetrazine. Also provided is a compound as defined above, or a conjugate as defined above, or a composition as defined above, for use as a medicament, wherein preferably at least one of R^{a}, R^{b}, R^{c}, and R^{d} is a pharmaceutically active substance, wherein preferably at least one of R^{a}, R^{b}, R^{c}, and R^{d} is a targeting moiety, wherein the pharmaceutically active substance is preferably a chemotherapeutic agent, and the targeting moiety is preferably an antibody or a fragment thereof.

The invention further provides a method for releasing a a small organic molecule or a pharmaceutically active substance, the method comprising the steps of: i) providing a conjugate as defined above wherein at least one of R^{a}, R^{b}, R^{c}, and R^{d} is the small organic molecule or pharmaceutically active substance; ii) contacting the provided conjugate with a 1,2,4,5-tetrazine; wherein the conjugate as defined above is preferably of general formula (II-a) or (VI-a) or (VII-O) or (VII-OH), more preferably of general formula (VI-a) or (VII-O) or (VII-OH), even more preferably of general formula (VII-O) or (VII-OH), and most preferably of general formula (VII-O).

### DESCRIPTION OF EMBODIMENTS

### Compound

The invention relates to compounds of general formula (I), or a salt thereof:

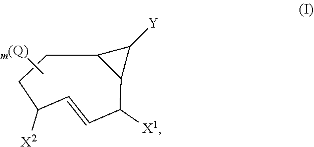

wherein
one of X¹ and X² is —R^{a} or —Z¹—C(═Z²)—R^{b}, and the other is —H;
Y is chosen from —C(═Z³)—R^{c} or —CH₂—R^{d};
Z¹, Z², and Z³ are in each instance chosen independently from O, S or NQ^{N};
R^{a}, R^{b}, R^{c}, and R^{d} are in each instance chosen independently from —O—Pr, —S—Pr, —NQ^{N1}Q^{N2}, a leaving group, a small organic molecule, a pharmaceutically active substance, or a targeting moiety; optionally in each instance chosen independently from —O—Pr, —S—Pr, —NQ^{N1}Q^{N2}, a leaving group, a pharmaceutically active substance, or a targeting moiety;
Pr is in each instance chosen independently from —H, a linear, branched, or cyclic C1-6 alkyl or acyl, or a protecting group, wherein acyl or alkyl are optionally unsaturated and optionally substituted with halogen, alkoxy, —C(═O)O(CH₂)₀₋₄H, or haloalkoxy; preferable optional substitutions are halogen, alkoxy, or haloalkoxy; a preferred —C(═O)O(CH₂)₀₋₄H is —C(═O)OCH₃. Q, Q^{N}, Q^{N1}, Q^{N2} are in each instance chosen independently from —H or a linear, branched or cyclic C1-6 alkyl, wherein alkyl is optionally unsaturated and optionally substituted with halogen, alkoxy, or haloalkoxy; and
m is 0, 1, 2, 3 or 4.
   Such a compound is called a compound according to the invention in the context of this application.

Compounds according to the invention comprise a (4E)-bicyclo[6.1.0]non-4-ene moiety. In other words, the carbon-carbon double bond depicted in general formulae (1), (II-a), (II-b), (II-c), (II-d), (II-e), (II-f), (II-g), (II-h), (II-i), (II-j), (III-a), (III-b), (III-c), (III-d), (III-e), (III-f), (III-g), (III-h), (IV-0), (IV-1a), (IV-1b), (IV-2a), (IV-2b), (IV-2c), (IV-3a), (IV-3b), (IV-4), (V-OO), (V-HO), (V-OH), (V-HH), (VI-a), (VI-b), (VII-OO), (VII-HO), (VII-OH), (VII-HH), (VIII-OO), (VIII-HO), (VIII-OH), and (VIII-HH) has an E configuration. Compounds according to the invention can be considered bi-functionalized trans-cyclooctenes, at least because Y and one of the X moieties can both allow functionalisation, or can both represent functionalisation.

X¹ and X² Moieties of the Compound

Compounds according to the invention comprise moieties X¹ and X² attached to the eight-membered ring in general formula (I). One of X¹ and X² is —R^{a} or —Z¹—C(═Z²)—R^{b}, and the other is —H. Because only one of X¹ and X² is not H, these moieties can sometimes together be referred to as X. Most preferably it is X¹ that is H and X² that is as defined elsewhere. Herein, Z¹ and Z² are in each instance chosen independently from O, S or NQ^{N}; R^{a} and R^{b} are in each instance chosen independently from —O—Pr, —S—Pr, —NQ^{N1}Q^{N2}, a leaving group, a small organic molecule, a pharmaceutically active substance, or a targeting moiety; Pr is in each instance chosen independently from —H, a linear, branched, or cyclic C1-6 alkyl or acyl, or a protecting group, wherein acyl or alkyl are optionally unsaturated and optionally substituted with halogen, alkoxy, or haloalkoxy, and optionally —C(═O)O(CH₂)₀₋₄H; and Q^{N}, Q^{N1} and Q^{N2} are in each instance chosen independently from —H or a linear, branched or cyclic C1-6 alkyl, wherein alkyl is optionally unsaturated and optionally substituted with halogen, alkoxy, or haloalkoxy, and optionally —C(═O)O(CH₂)₀₋₄H.

When comprised in X¹ or X², C1-6 alkyl can be C1-4 alkyl, more preferably C2-4 alkyl, most preferably C2 alkyl. When comprised in X¹ or X², the alkyl is preferably not unsaturated. When comprised in X¹ or X², the alkyl is preferably not optionally substituted.

In preferred embodiments, X¹ is —R^{a} or —Z¹—C(═Z²)—R^{b} and X² is —H. More preferably, m is 0 in these preferred embodiments.

In preferred embodiments, X² is —R^{a} or —Z¹—C(═Z²)—R^{b} and X¹ is —H. More preferably, m is 0 in these preferred embodiments.

In preferred embodiments, X¹ is —Z¹—C(═Z²)—R^{b} and X² is —H, wherein Z¹ and Z² are in each instance chosen independently from O, S or NQ^{N}, more preferably from O and S, even more preferably Z¹ and Z² are O. Most preferably, m is 0 in these preferred embodiments.

In preferred embodiments, X² is —Z¹—C(═Z²)—R^{b} and X¹ is —H, wherein Z¹ and Z² are in each instance chosen independently from O, S or NQ^{N}, more preferably from O and S, even more preferably Z¹ and Z² are O. Most preferably, m is 0 in these embodiments.

In preferred embodiments, X¹ is —Z¹—C(═Z²)—R^{b}, wherein —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b}. More preferably, m is 0 in these embodiments.

In preferred embodiments, X² is —Z¹—C(═Z²)—R^{b}, wherein —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b}. More preferably, m is 0 in these embodiments.

In preferred embodiments, one of X¹ and X² is —Z¹—C(═Z²)—R^{b}, and the other is —H, wherein R^{b} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Even more preferably, —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b} in these preferred embodiments. Most preferably, m is 0 and/or X¹ is —H in these preferred embodiments.

As used herein, a protecting group has its customary meaning of a group that is linked to the heteroatom in such a way as to reduce its reactivity, or to protect it from certain conditions. After having served their function, protecting groups can generally be removed again using routine chemistry. A skilled person is well aware of protecting groups and can select useful protecting groups for particular groups or atoms to be protected, reaction conditions, storage conditions, purification techniques, or available deprotection techniques. A helpful reference in this regard is Greene's Protective Groups in Organic Synthesis, Wuts & Greene, DOI:10.1002/0470053488.

In preferred embodiments, one of X¹ and X² is —Z¹—C(═Z²)—R^{b}, and the other is —H, wherein R^{b} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Even more preferably, —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b} in these preferred embodiments. Most preferably, m is 0 and/or X¹ is H in these preferred embodiments.

Leaving groups have their customary meaning here. A skilled person is capable of selecting appropriate leaving groups. Preferred leaving groups leave as anions via heterolysis, and can stabilize the additional electron density that results from bond heterolysis. Preferred leaving groups are active esters and halogens, and preferred active esters are (halo)phenyl ester such as pentafluorophenyl ester, N-hydroxysuccinimidyl ester, thio(halo)phenyl ester, and nitrophenyl ester such as paranitrophenyl ester.

In preferred embodiments, the leaving group is (LG-1), (LG-2), (LG-3), (LG-4), (LG-5), (LG-6), (LG-7), or (LG-8):

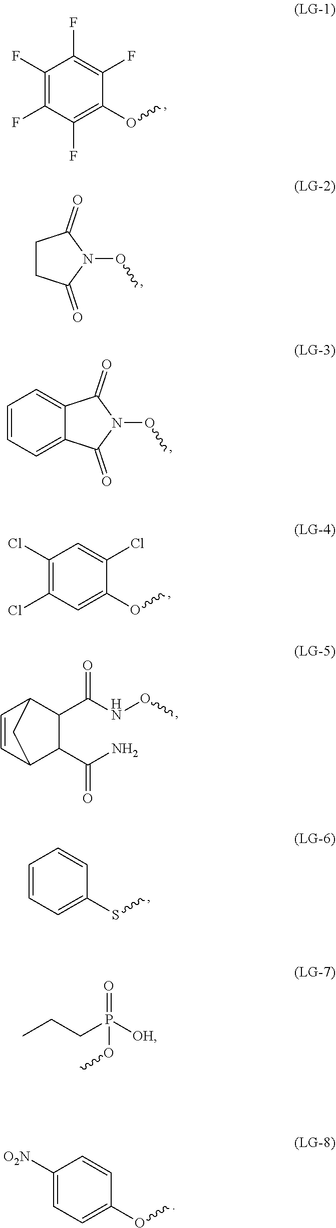

The above leaving groups are particularly suited for R^{b} and R^{c}. For R^{a} and R^{d}, leaving groups are preferably linked via a carbonyl moiety, for instance via —O—C(═O)—P′, wherein P′ represents for instance a moiety as depicted above.

Particularly preferred are LG-1, LG-2, and LG-8, even more so LG-1 and LG-2, and LG-1 is most preferred.

In preferred embodiments, one of X¹ and X² is —Z¹—C(═Z²)—R^{b}, and the other is —H, wherein R^{b} is chosen from —O—Pr, —S—Pr and a leaving group, more preferably from —O—Pr and a leaving group, even more preferably from —O—H and a leaving group. Here, most preferably, m is 0 and/or X¹ is H.

In preferred embodiments, X¹ is —R^{a} and X² is —H, wherein more preferably m is 0. In other preferred embodiments, X² is —R^{a} and X¹ is —H, wherein more preferably m is 0. In preferred embodiments, one of X¹ and X² is —R^{a}, and the other is —H, wherein R^{a} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Most preferably, m is 0 and/or X¹ is —H in these preferred embodiments.

In preferred embodiments, one of X¹ and X² is —R^{a}, and the other is —H, wherein R^{a} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Most preferably, m is 0 and/or X¹ is H in these preferred embodiments.

In preferred embodiments, one of X¹ and X² is —R^{a}, and the other is —H, wherein R^{a} is chosen from —O—Pr, —S—Pr and a leaving group, more preferably from —O—Pr and a leaving group, even more preferably from —O—H and a leaving group. Most preferably, m is 0 and/or X¹ is H in these preferred embodiments.

In preferred embodiments, the compounds according to the invention are of general formula (II-a), (II-b), (II-c), (II-d), (II-e), (II-f), (II-g), (II-h), (II-i), or (II-j), or enantiomers thereof, preferably wherein m is 0 and/or X¹ is —H:

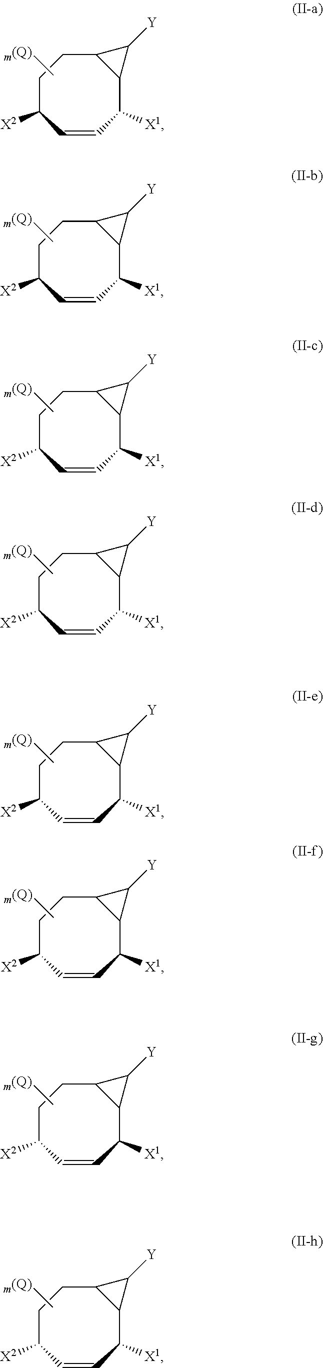

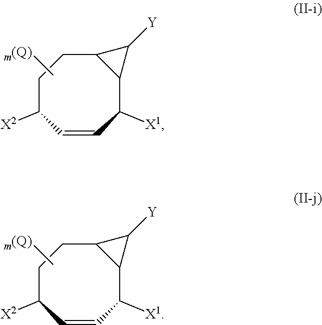

Of the above, II-a, II-b, II-c, II-d, and II-j are preferred. In other embodiments, the compound is of one of formulas II-e, II-f, II-g, II-h, or II-i. Of the above, II-a and II-b and II-j are most preferred, wherein II-a and II-b are more preferred and II-a is most preferred.

In preferred embodiments, the compounds according to the invention are of general formula (II-a), wherein one of X¹ and X² is —Z¹—C(═Z²)—R^{b}, and the other is —H, wherein Z¹ and Z² are in each instance chosen independently from O, S or NQ^{N}, more preferably from O and S, even more preferably Z¹ and Z² are O. Most preferably, m is 0 and/or X¹ is —H in these preferred embodiments.

In preferred embodiments, the compounds according to the invention are of general formula (II-a), wherein one of X¹ and X² is —Z¹—C(═Z²)—R^{b}, and the other is —H, wherein —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b}. More preferably, m is 0 and/or X¹ is —H in these preferred embodiments.

In preferred embodiments, the compounds according to the invention are of general formula (II-a), wherein one of X¹ and X² is —Z¹—C(═Z²)—R^{b}, and the other is —H, wherein R^{b} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Even more preferably, —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b} in these preferred embodiments. Most preferably, m is 0 and/or X¹ is —H in these preferred embodiments.

In preferred embodiments, the compounds according to the invention are of general formula (II-a), wherein one of X¹ and X² is —Z¹—C(═Z²)—R^{b}, and the other is —H, wherein R^{b} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Even more preferably, —Z¹—C(═Z²)—R^{b} is chosen from —O—C(═O)—R^{b}, —S—C(═O)—R^{b}, —O—C(═S)—R^{b}, —S—C(═S)—R^{b}, —NQ^{N}—C(═O)—R^{b}, —NQ^{N}—C(═S)—R^{b}, —NH—C(═O)—R^{b}, and —NH—C(═S)—R^{b} in these preferred embodiments. Most preferably, m is 0 and/or X¹ is H in these preferred embodiments.

In preferred embodiments, the compounds according to the invention are of general formula (II-a), wherein one of X¹ and X² is —Z¹—C(═Z^{a})—R^{b}, and the other is —H, wherein R^{b} is chosen from —O—Pr, —S—Pr and a leaving group, more preferably from —O—Pr and a leaving group, even more preferably from —O—H and a leaving group. Most preferably, m is 0 and/or X¹ is H in these preferred embodiments.

In preferred embodiments, the compounds according to the invention are of general formula (II-a), wherein one of X¹ and X² is —R^{a}, and the other is —H, more preferably wherein m is 0 and/or X¹ is —H.

In preferred embodiments, the compounds according to the invention are of general formula (II-a), wherein one of X¹ and X² is —R^{a}, and the other is —H, wherein R^{a} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Most preferably, m is 0 and/or X¹ is —H in these preferred embodiments.

In preferred embodiments, the compounds according to the invention are of general formula (II-a), wherein one of X¹ and X² is —R^{a}, and the other is —H, wherein R^{a} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Most preferably, m is 0 and/or X¹ is —H in these preferred embodiments.

In preferred embodiments, the compounds according to the invention are of general formula (II-a), wherein one of X¹ and X² is —R, and the other is —H, wherein R^{a} is chosen from —O—Pr, —S—Pr and a leaving group, more preferably from —O—Pr and a leaving group, even more preferably from —O—H and a leaving group. Most preferably, m is 0 and/or X¹ is H in these preferred embodiments.

In preferred embodiments—NQ^{N1}Q^{N2} is —N(CH₃)(CH₂C(═O)OCH₃) or —NHCH₂C(═O)OCH₃.

### Y Moiety of the Compound

Compounds according to the invention comprise moiety Y attached to the three-membered ring in general formula (I). Y is chosen from —C(═Z³)—R^{c} or —CH₂—R^{d}. A skilled person will appreciate that the latter moiety is easily synthetically accessible from the former moiety, for instance through reduction when Z³ is O. Herein, Z³ is chosen from O, S or NQ^{N}; R^{c} is chosen from —O—Pr, —S—Pr, —NQ^{N1}Q^{N2}, a leaving group, a small organic molecule, a pharmaceutically active substance, or a targeting moiety; Pr is in each instance chosen independently from —H, a linear, branched, or cyclic C1-6 alkyl or acyl, or a protecting group, wherein acyl or alkyl are optionally unsaturated and optionally substituted with halogen, alkoxy, or haloalkoxy, and optionally —C(═O)O(CH₂)₀₋₄H; and NQ^{N1}Q^{N2} and Q^{N2} are in each instance chosen independently from —H or a linear, branched or cyclic C1-6 alkyl, wherein alkyl is optionally unsaturated and optionally substituted with halogen, alkoxy, or haloalkoxy, and optionally —C(═O)O(CH₂)₀₋₄H.

When comprised in Y, C1-6 alkyl can be C1-4 alkyl, more preferably C2-4 alkyl, most preferably C2 alkyl. When comprised in Y, the alkyl is preferably not unsaturated. When comprised in Y, the alkyl is preferably not optionally substituted.

In preferred embodiments, Y is —C(═Z³)—R^{c}. More preferably, m is 0 and/or X¹ is —H in these preferred embodiments.

In preferred embodiments, Y is —C(═Z³)—R^{c}, wherein Z³ is chosen from O, S or NO, more preferably from O and S, even more preferably Z³ is O. Most preferably, m is 0 and/or X¹ is —H in these preferred embodiments.

In preferred embodiments, Y is —C(═Z³)—R^{c}, wherein R^{c} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Even more preferably, Z³ is chosen from O and S in these preferred embodiments. Most preferably, m is 0 and/or X¹ is —H in these preferred embodiments.

In preferred embodiments, Y is —C(═Z³)—R^{c}, wherein R^{c} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Even more preferably, Z³ is chosen from O and S in these preferred embodiments. Most preferably, m is 0 and/or X¹ is —H in these preferred embodiments.

In preferred embodiments, Y is —C(═Z³)—R^{c}, wherein R^{c} is chosen from —O—Pr, —S—Pr and a leaving group, more preferably from —O—Pr and a leaving group, even more preferably from —O—H and a leaving group. Even more preferably, Z³ is chosen from O and S in these preferred embodiments. Most preferably, m is 0 and/or X¹ is —H in these preferred embodiments.

In preferred embodiments, Y is —CH₂R^{d}. More preferably, m is 0 and/or X¹ is H in these preferred embodiments.

In preferred embodiments, Y is —CH₂—R^{d}, wherein R^{d} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Most preferably, m is 0 and/or X¹ is —H in these preferred embodiments.

In preferred embodiments, Y is —CH₂—R^{d}, wherein R^{d} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Most preferably, m is 0 and/or X¹ is —H in these preferred embodiments.

In preferred embodiments, Y is —CH₂—R^{d}, wherein R^{d} is chosen from —O—Pr, —S—Pr and a leaving group, more preferably from —O—Pr and a leaving group, even more preferably from —O—H and a leaving group. Most preferably, m is 0 and/or X¹ is —H in these preferred embodiments.

In preferred embodiments, the compounds according to the invention are of general formula (III-a), (III-b), (III-c), (III-d), (III-e), (III-f), (III-g), or (III-h), preferably wherein m is 0 and/or X¹ is —H:

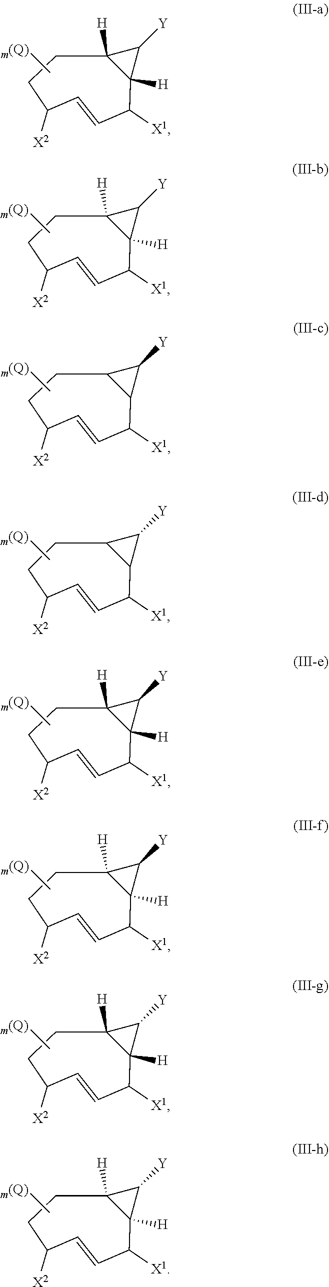

Of the above, III-a, III-c, and III-e are preferred. In other embodiments, the compound is of one of formulas III-b, III-d, III-f, III-g, or III-h. Of the above, III-a and III-e are highly preferred, wherein III-e is most preferred. As used herein, when a compound is of a general formula wherein stereochemistry is indicated, reference to that stereochemistry is primarily intended. A compound of general formula III-a is thus primarily characterised by the stereochemistry of the two bridgehead hydrogen atoms. It should be clear that a compound can be of both general formula III-e and II-a, for instance—a combination that is particularly preferred.

In preferred embodiments, the compounds according to the invention are of general formula (III-e), wherein Y is —C(═Z³)—R^{c}. More preferably, m is 0 and/or X¹ is —H in these preferred embodiments.

In preferred embodiments, the compounds according to the invention are of general formula (III-e), wherein Y is —C(═Z³)—R^{b}, wherein Z³ is chosen from O, S or NQ^{N}, more preferably from O and S, even more preferably Z³ is O. Most preferably, m is 0 and/or X¹ is —H in these preferred embodiments.

In preferred embodiments, the compounds according to the invention are of general formula (III-e), wherein Y is —C(═Z³)—R^{c}, wherein R^{c} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Even more preferably, Z³ is chosen from O and S in these preferred embodiments. Most preferably, m is 0 and/or X¹ is —H in these preferred embodiments.

In preferred embodiments, the compounds according to the invention are of general formula (III-e), wherein Y is —C(═Z³)—R^{c}, wherein R^{c} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Even more preferably, Z³ is chosen from O and S in these preferred embodiments. Most preferably, m is 0 and/or X¹ is —H in these preferred embodiments.

In preferred embodiments, the compounds according to the invention are of general formula (III-e), wherein Y is —C(═Z³)—R^{c}, wherein Re is chosen from —O—Pr, —S—Pr and a leaving group, more preferably from —O—Pr and a leaving group, even more preferably from —O—H and a leaving group. Even more preferably, Z³ is chosen from O and S in these preferred embodiments. Most preferably, m is 0 and/or X¹ is —H in these preferred embodiments.

In preferred embodiments, the compounds according to the invention are of general formula (III-e), wherein Y is —CH₂—R^{d}. More preferably, m is 0 and/or X¹ is H in these preferred embodiments.

In preferred embodiments, the compounds according to the invention are of general formula (III-e), wherein Y is —CH₂—R^{d}, wherein R^{d} is chosen from —O—Pr and —S—Pr, preferably wherein Pr a protecting group, more preferably wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE). Most preferably, m is 0 and/or X¹ is —H in these preferred embodiments.

In preferred embodiments, the compounds according to the invention are of general formula (III-e), wherein Y is —CH₂R^{d}, wherein R^{d} is a leaving group, more preferably wherein the leaving group is a halogen, preferably chlorine, bromine, or iodine, more preferably chlorine, or wherein the leaving group is (LG-1), (LG-2), (LG-3), (LG-4), (LG-5), (LG-6), (LG-7), or (LG-8). Most preferably, m is 0 and/or X¹ is —H in these preferred embodiments.

In preferred embodiments, the compounds according to the invention are of general formula (III-e), wherein Y is —CH₂R^{d}, wherein R^{d} is chosen from —O—Pr, —S—Pr and a leaving group, more preferably from —O—Pr and a leaving group, even more preferably from —O—H and a leaving group. Most preferably, m is 0 and/or X¹ is —H in these preferred embodiments.

### (Q)ₘ Moiety of the Compound

Compounds according to the invention may comprise 0, 1, 2, 3 or 4 Q moieties attached to the eight-membered ring in general formula (I). The number of Q moieties attached to the eight-membered ring is m.

In preferred embodiments, the compounds according to the invention are of general formula (IV-0), (IV-1a), (IV-1b), (IV-2a), (IV-2b), (IV-2c), (IV-3a), (IV-3b), or (IV-4), preferably wherein X¹ is —H:

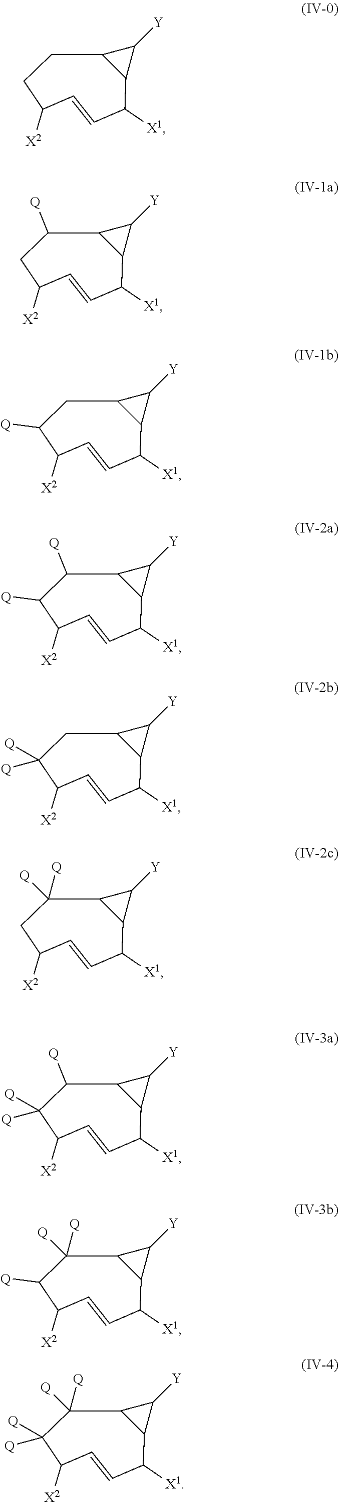

In preferred embodiments, the compounds according to the invention are of general formula (IV-1a), (IV-1b), (IV-2a), (IV-2b), (IV-2c), (IV-3a), (IV-3b), or (IV-4), wherein Q is in each instance chosen independently from a linear, branched or cyclic C1-6 alkyl, wherein alkyl is optionally unsaturated and optionally substituted with halogen, alkoxy, or haloalkoxy, preferably wherein X¹ is —H.

In some embodiments, m is 0, 1, 2, or 3. In some embodiments, m is 1, 2, or 3. In some embodiments, m is 1 or 2. In some preferred embodiments, m is 0 or 1. In some embodiments, m is 1. In some embodiments, m is 2. Most preferably m is 0.

### Preferred Substitutions and Configurations of the Compound

In preferred embodiments, one of X¹ and X² is —Z¹—C(═Z²)—R^{b}, and the other of X¹ and X² is —H, and Y is —C(═Z³)—R^{c}. More preferably, m is 0 and/or X¹ is —H in these preferred embodiments.

In preferred embodiments, one of X¹ and X² is —Z¹—C(═Z²)—R^{b}, and the other of X¹ and X² is —H, and Y is —CH₂—R^{d}. More preferably, m is 0 and or X¹ is —H in these preferred embodiments.

In preferred embodiments, one of X¹ and X² is —R^{a}, and the other of X¹ and X² is —H, and Y is —C(═Z³)—R^{c}. More preferably, m is 0 and/or X¹ is —H in these preferred embodiments.

In preferred embodiments, one of X¹ and X² is —R^{a}, and the other of X¹ and X² is —H, and Y is —CH₂—R^{d}. More preferably, m is 0 and/or X¹ is —H in these preferred embodiments.

In preferred embodiments, one of X¹ and X² is —Z¹—C(═Z²)—R^{b}, and the other of X¹ and X² is —H, and Y is —C(═Z³)—R^{c}, wherein Z¹, Z², and Z² are in each instance chosen independently from O, S or NQ^{N}, more preferably from O and S, even more preferably Z¹, Z², and Z² are O. Most preferably, m is 0 and/or X¹ is —H in these preferred embodiments.

In preferred embodiments, one of X¹ and X² is —Z¹—C(═Z²)—R^{b}, and the other of X¹ and X² is —H, and Y is —CH₂—R^{d}, wherein Z¹, Z², and Z³ are in each instance chosen independently from O, S or NQ^{N}, more preferably from O and S, even more preferably Z¹, Z², and Z² are O. Most preferably, m is 0 and/or X¹ is —H in these preferred embodiments.

In preferred embodiments, one of X¹ and X² is —R^{a}, and the other of X¹ and X² is —H, and Y is —C(═Z³)—R^{c}, wherein Z¹, Z², and Z³ are in each instance chosen independently from O, S or NQ^{N}, more preferably from O and S, even more preferably Z¹, Z², and Z² are O. Most preferably, m is 0 and/or X¹ is —H in these preferred embodiments.

In preferred embodiments, X² is —O—C(═O)—R^{b}, and Y is —C(═O)—R¹. More preferably, m is 0 in these preferred embodiments and the compound is thus of general formula (V-OO). Most preferably, R^{b} and R^{c} are in each instance chosen independently from —O—Pr, —S—Pr and a leaving group, or from —O—Pr and a leaving group, or from —O—H and a leaving group.

In preferred embodiments, X² is —C(═O)—R^{b}, and Y is —CH₂—R^{d}. More preferably, m is 0 in these preferred embodiments and the compound is thus of general formula (V-OH). Most preferably, R^{b} and R^{d} are in each instance chosen independently from —O—Pr, —S—Pr and a leaving group, or from —O—Pr and a leaving group, or from —O—H and a leaving group.

In preferred embodiments, X² is —R^{a}, and Y is —C(═O)—R^{c}. More preferably, m is 0 in these preferred embodiments and the compound is thus of general formula (V-HO). Most preferably, R^{a} and R^{c} are in each instance chosen independently from —O—Pr, —S—Pr and a leaving group, or from —O—Pr and a leaving group, or from —O—H and a leaving group.

In preferred embodiments, X² is —R^{a}, and Y is —CH₂—R^{d}. More preferably, m is 0 in these preferred embodiments and the compound is thus of general formula (V-HH). Most preferably, R^{a} and R^{d} are in each instance chosen independently from —O—Pr, —S—Pr and a leaving group, or from —O—Pr and a leaving group, or from —O—H and a leaving group.

In preferred embodiments, the compounds according to the invention are of general formula (V-OO), (V-HO), V(OH) or (V-HH):

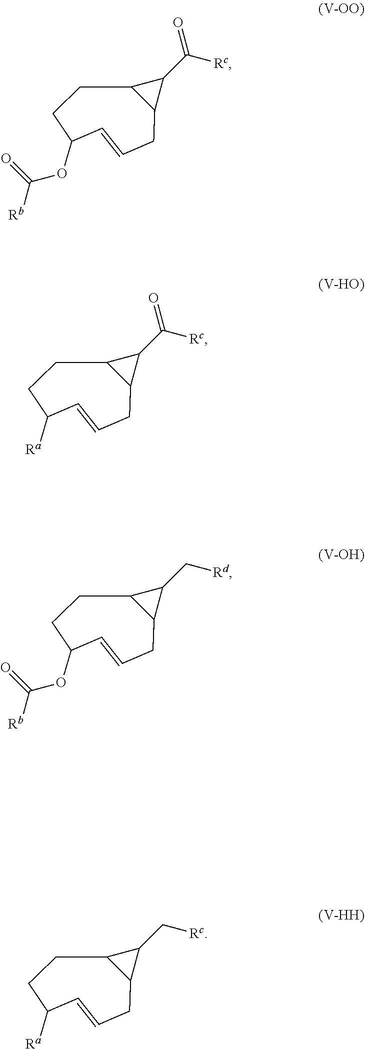

In preferred embodiments, the compounds of the invention are of general formula (II-a), (II-b), (II-c), (II-d), (II-e), (II-f), (II-g), (II-h), (II-i), (II-j), (III-a), (III-b), (III-c), (III-d), (III-e), (III-f), (III-g), (III-h), (IV-0), (IV-1a), (IV-1b), (IV-2a), (IV-2b), (IV-2c), (IV-3a), (IV-3b), (IV-4), (V-OO), (V-HO), (V-OH), (V-HH), (VI-a), (VI-b), (VII-OO), (VII-HO), (VII-OH), (VII-HH), (VIII-OO), (VIII-HO), (VIII-OH), or (VIII-HH), wherein at least one of R^{a}, R^{b}, R^{c}, and R^{d} is a small organic molecule or a pharmaceutically active substance and/or wherein at least one of R^{a}, R^{b}, R^{c}, and R^{d} is a targeting moiety, more preferably wherein the pharmaceutically active substance is a chemotherapeutic agent, more preferably wherein and the targeting moiety is preferably an antibody or a fragment thereof. Most preferably, compounds according to these preferred embodiments are for use a medicament, preferably for use in the treatment or diagnosis of cancer.

In preferred embodiments, the compounds according to the invention are of general formula (II-a) and (III-a), or (II-a) and (III-b), or (II-a) and (III-c), or (II-a) and (III-d), or (II-a) and (III-e), or (II-a) and (III-f), or (II-a) and (III-g), or (II-a) and (III-h), or (II-b) and (III-a), or (II-b) and (III-b), or (II-b) and (III-c), or (II-b) and (III-d), or (II-b) and (III-e), or (II-b) and (III-f), or (II-b) and (III-g), or (II-b) and (III-h), or (II-c) and (III-a), or (II-c) and (III-b), or (II-c) and (III-c), or (II-c) and (III-d), or (II-c) and (III-e), or (II-c) and (III-f), or (II-c) and (III-g), or (II-c) and (III-h), or (II-d) and (III-a), or (II-d) and (III-b), or (II-d) and (III-c), or (II-d) and (III-d), or (II-d) and (III-e), or (II-d) and (III-f), or (II-d) and (III-g), or (II-d) and (III-h), or (II-e) and (III-a), or (II-e) and (III-b), or (II-e) and (III-c), or (II-e) and (III-d), or (II-e) and (III-e), or (II-e) and (III-f), or (II-e) and (III-g), or (II-e) and (III-h), or (II-f) and (III-a), or (II-f) and (III-b), or (II-f) and (III-c), or (II-f) and (III-d), or (II-f) and (III-e), or (II-f) and (III-f), or (II-f) and (III-g), or (II-f) and (III-h), or (II-g) and (III-a), or (II-g) and (III-b), or (II-g) and (III-c), or (II-g) and (III-d), or (II-g) and (III-e), or (II-g) and (III-f), or (II-g) and (III-g), or (II-g) and (III-h), or (II-h) and (III-a), or (II-h) and (III-b), or (II-h) and (III-c), or (II-h) and (III-d), or (II-h) and (III-e), or (II-h) and (III-f), or (II-h) and (III-g), or (II-h) and (III-h), or (II-i) and (III-a), or (II-i) and (III-b), or (II-i) and (III-c), or (II-i) and (III-d), or (II-i) and (III-e), or (II-i) and (III-f), or (II-i) and (III-g), or (II-i) and (III-h), or (II-j) and (III-a), or (II-j) and (III-b), or (II-j) and (III-c), or (II-j) and (III-d), or (II-j) and (III-e), or (II-j) and (III-f), or (II-j) and (III-g), or (II-j) and (III-h), or enantiomers thereof, preferably wherein m is 0 and/or X¹ is —H. Herein, it is understood that “a compound of general formula (XX) and (YY)” should be interpreted as a compound which can be represented by both general formula (XX) and general formula (YY).

In preferred embodiments, the compounds according to the invention are of general formula (VI-a) or (VI-b) or an enantiomer thereof, more preferably wherein m is 0 and/or X¹ is —H:

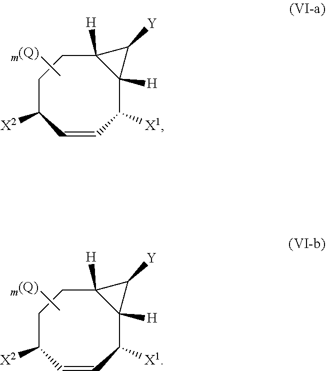

In preferred embodiments, the compounds according to the invention are of general formula (VII-OO), (VII-HO), (VII-OH), (VII-HH), (VIII-OO), (VIII-HO), (VIII-OH), or (VIII-HH), or an enantiomer thereof, more preferably wherein R^{a}, R^{b}, R^{c} and R^{d} are in each instance chosen independently from —O—Pr, —S—Pr and a leaving group, even more from —O—Pr and a leaving group, most preferably from —O—H and a leaving group:

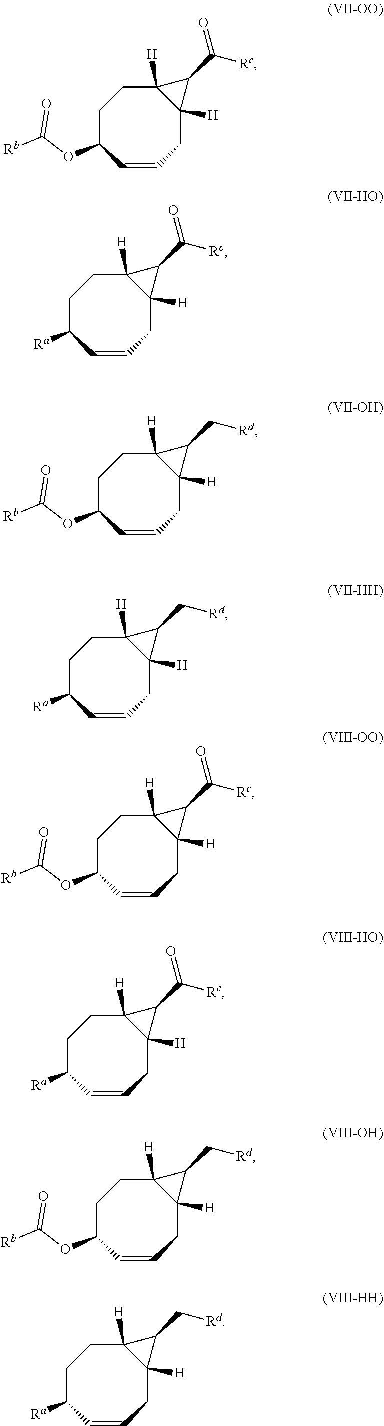

In preferred embodiments, the compounds according to the invention are of general formula (VII-OO), (VII-HO), (VII-OH), or (VII-HH), or an enantiomer thereof, more preferably wherein R^{a}, R^{b}, R^{c} and R^{d} are in each instance chosen independently from —O—Pr, —S—Pr and a leaving group, even more from —O—Pr and a leaving group, most preferably from —O—H and a leaving group.

In preferred embodiments, when stereochemistry is indicated for atoms in the double bond of the cyclooectene ring or adjacent to that double bond, reference is made to both the structure as drawn and its enantiomer. A skilled person will appreciate that stereocenters in compounds of the invention can have an effect on the steric or electronic organisation of these compounds, yet that full mirror images (enantiomers) lead to compounds with identical steric and electronic organisation.

### Click and Release

In preferred embodiments, a compound according to the invention is able to undergo a click reaction, more preferably wherein the click reaction is between the compound and a 1,2,4,5-tetrazine, most preferably a tetrazine of general formula (Tz), wherein Q^{Tz} is in each instance chosen independently from —H, phenyl, 2-pyridyl, 3-pyridyl, 4-pyridyl, 2,6-pyrimidyl, 2,5-pyrimidyl, 3,5-pyrimidyl, and 2,4-pyrimidyl.

In preferred embodiments, Q^{Tz} is in each instance chosen independently from 2-pyridyl, 3-pyridyl, 4-pyridyl. In more preferred embodiments, Q^{Tz} is 4-pyridyl.

In preferred embodiments, a compound according to the invention is able to undergo a click reaction, wherein the click reaction can be represented by the general scheme (I)+(Tz)→(I-Tz):

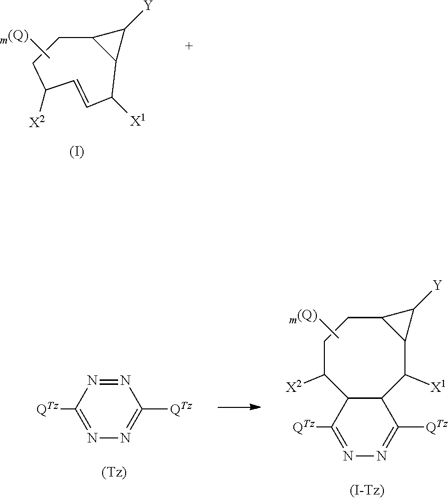

In preferred embodiments, the rate of the click reaction is at least 50 M⁻¹s⁻¹, 100 M⁻¹s⁻¹, 150 M⁻¹s⁻¹, or 200 M⁻¹s⁻¹ for a 10 μM concentration of (I) at 20° C., preferably under the conditions specified in Example 4.

In preferred embodiments, the rate of the click reaction is at least 250 M⁻¹s⁻¹, 300 M⁻¹s⁻¹, 350 M⁻¹s⁻¹, or 400 M⁻¹s⁻¹ for a 50 μM concentration of (I) at 20° C., preferably under the conditions specified in Example 4.

In preferred embodiments, a product of the click reaction of general formula (I-Tz) is ale to undergo a release reaction, wherein X² or a part thereof is detached from the rest of (I-Tz), more preferably wherein said part is a leaving group.

In preferred embodiments, the release reaction can be represented by general scheme (I-Tz)→X^{2 #}+(I-Tz^{#}) wherein X^{2 #}represents one or more moieties derived from X²

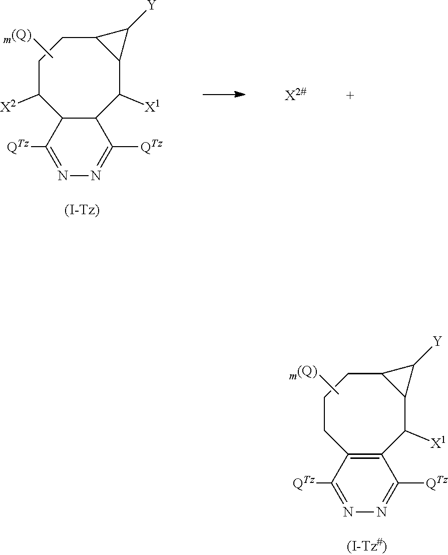

In some embodiments the release is of X¹ instead.

For I-Tz it is preferred that X¹ or X² is —Z¹—C(═Z²)—R^{b}, preferably that X¹ or X² comprises a carbamate or a carbonate. In such embodiments, the release of X2 releases R^{b} under loss of Z¹═C═Z², which when Z¹ and Z² are O equates to loss of CO₂. It is therefore highly preferred that X¹ or X² is —O—C(═O)—R^{b}, most preferably X² is —O—C(═O)—R^{b}.

In preferred embodiments, the release reaction is spontaneous at 20° C., preferably under the conditions specified in Example 5, wherein the release reaction at 20° C. results in at least 95% conversion after 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes.

In preferred embodiments, the release reaction is spontaneous at 20° C., preferably under the conditions specified in Example 5, wherein the release reaction at 20° C. results in at least 60%, 65%, 700%, 75%, 800%, 85%, 900%, 95%, 96%, 97%, 98%, or 99% conversion after 20 minutes.

In preferred embodiments, a compound according to the invention is of general formula (V-OO), and is able to undergo a click reaction, represented by the general scheme scheme (V-OO)→(Tz)→(V-OO-Tz), more preferably wherein (V-OO-Tz) is able to undergo a spontaneous release reaction, represented by general scheme (V-OO-Tz)→R^{b}+CO₂+(V-OO-Tz^{#}), most preferably wherein the release reaction at 20° C. results in at least 95% conversion after 20 minutes:

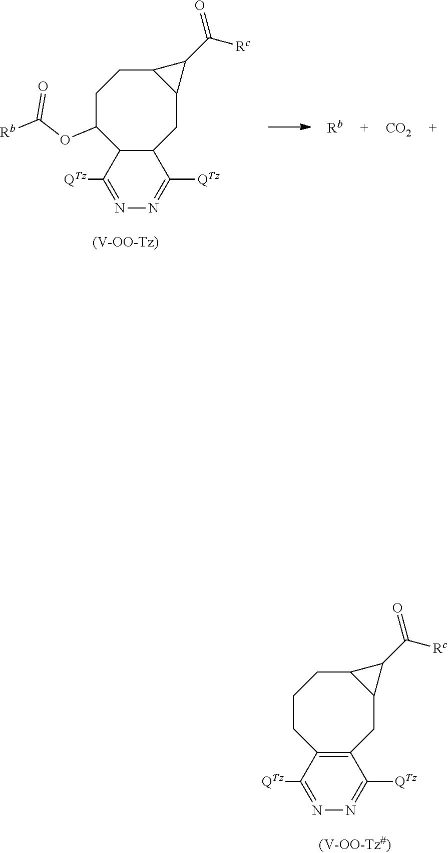

In preferred embodiments, a compound according to the invention is of general formula (V-OH), and is able to undergo a click reaction, represented by the general scheme scheme (V-OH)+(Tz)→(V-OH-Tz), more preferably wherein (V-OH-Tz) is able to undergo a spontaneous release reaction, represented by general scheme (V-OH-Tz)→R^{b}+CO₂+(V-OH-Tz^{#}), most preferably wherein the release reaction at 20° C. results in at least 95% conversion after 20 minutes:

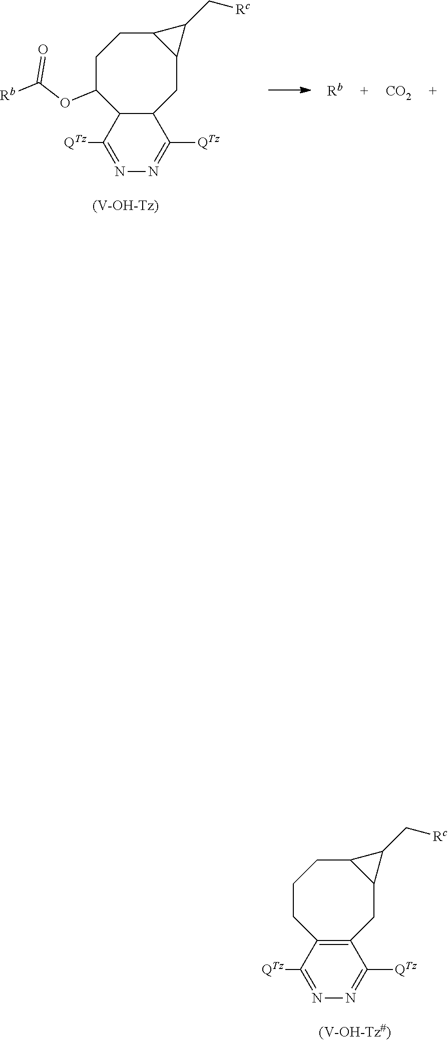

In preferred embodiments, a compound according to the invention is of general formula (VII-OO), and is able to undergo a click reaction, represented by the general scheme scheme (VII-OO)→(Tz)→(VII-OO-Tz), more preferably wherein (VII-OO-Tz) is able to undergo a spontaneous release reaction, represented by general scheme (VII-OO-Tz)→R^{b}+CO₂+(VII-OO-Tz^{#}), most preferably wherein the release reaction at 20° C. results in at least 95% conversion after 20 minutes:

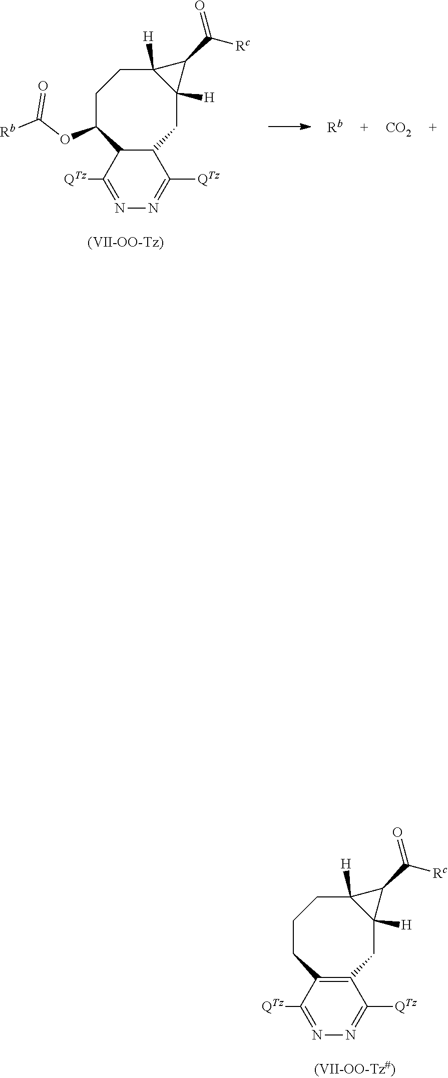

In preferred embodiments, a compound according to the invention is of general formula (VII-OH), and is able to undergo a click reaction, represented by the general scheme scheme (VII-OH)+(Tz)→(VII-OH-Tz), more preferably wherein (VII-OH-Tz) is able to undergo a spontaneous release reaction, represented by general scheme (VII-OH-Tz)→R^{b}+CO₂+(VII-OH-Tz^{#}), most preferably wherein the release reaction at 20° C. results in at least 95% conversion after 20 minutes:

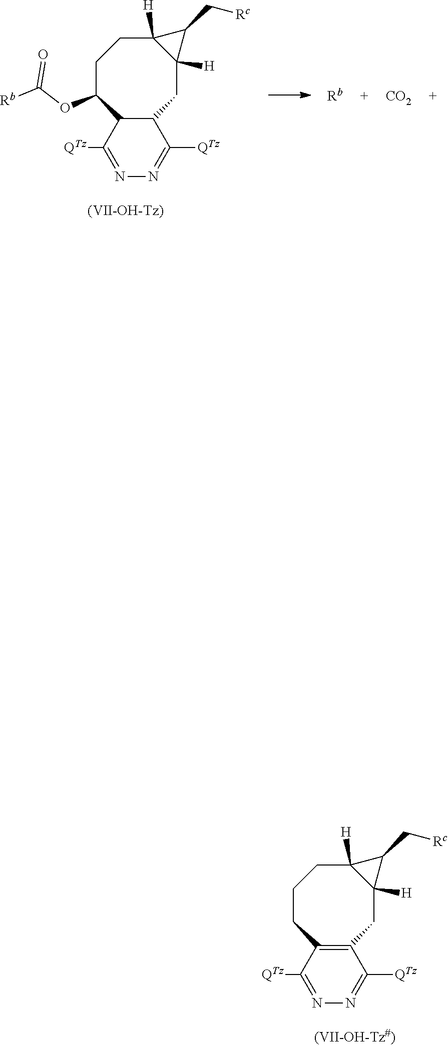

In the context of the release reaction, it is clear that an isolated R^{b}, as drawn in the general schemes above, is meant to include all relevant ions, salts and protonation forms or the R^{b} moiety defined as part of a general formula (I). Preferably, R^{b} is a leaving group in this context.

From the above, it follows that also provided is a method for releasing a a small organic molecule or a pharmaceutically active substance, the method comprising the steps of:
i) providing a conjugate according to the invention wherein at least one of R^{a}, R^{b}, R^{c}, and R^{d} is the a small organic molecule or the pharmaceutically active substance;
ii) contacting the provided conjugate with a 1,2,4,5-tetrazine;
wherein the conjugate as defined in claim 11 is preferably of general formula (II-a) or (VI-a) or (VII-O) or (VII-OH), more preferably of general formula (VI-a) or (VII-O) or (VII-OH), even more preferably of general formula (VII-O) or (VII-OH), and most preferably of general formula (VII-O). Preferably in these cases a small organic molecule or a pharmaceutically active compound is comprised in R^{a} or R^{b}, more preferably in R^{b}.

### Further Definitions

In the context of the invention, a salt of a compound according to the invention is preferably a pharmaceutically acceptable salt. Such salts include salts derived from inorganic bases such as Li, Na, K, Ca, Mg, Fe, Cu, Zn and Mn; salts of organic bases such as N, N′-diacetylethylenediamine, glucamine, triethylamine, choline, dicyclohexylamine, benzylamine, trialkylamine, thiamine, guanidine, diethanolamine, alpha-phenylethylamine, piperidine, morpholine, pyridine, hydroxyethylpyrrolidine, hydroxyethylpiperidine, and the like. Such salts also include amino acid salts such as glycine, alanine, cystine, cysteine, lysine, arginine, phenylalanine, guanidine, etc.

Such salts may include acid addition salts where appropriate, which are for example sulphates, nitrates, phosphates, perchlorates, borates, hydrohalides such as HCl or HBr salts, acetates, trifluoroacetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Preferred salts are HCl salts, formic acid salts, acetic acid salts, and trifluoroacetic acid salts. More preferred salts are HCl salts, acetic acid salts and formic acid salts, most preferably HCl salts.

The compound according to the invention can be a hydrate or a solvate. In the context of the invention a hydrate refers to a solvate wherein the solvent is water. The term solvate, as used herein, refers to a crystal form of a substance which contains solvent. Solvates are preferably pharmaceutically acceptable solvates and may be hydrates or may comprise other solvents of crystallization such as alcohols, ether, and the like.

In the context of this invention, the number of carbon atoms in a moiety such as a linear, branched, or cyclic alkyl, or acyl is indicated as for example C1-6, in this non-limiting case indicating that from 1 to 6 carbon atoms are envisaged, such as 1, 2, 3, 4, 5, or 6 carbon atoms. Similarly C2-4 alkyl has 2, 3, or 4 carbon atoms. The number of carbon atoms can be expressed as the total number of carbon atoms not counting further substitutions, the total number of carbon atoms, or as the number of carbon atoms that can be found in the longest continuous internal sequence of carbon atoms. Preferably, the number of carbon atoms is expressed as the total number of carbon atoms not counting further substitutions.

In the context of this invention, unsubstituted alkyl groups have the general formula CₙH₂ₙ₊₁ and may be linear or branched. Unsubstituted alkyl groups may also be cyclic, and thus have the concomitant general formula CₙH₂ₙ₊₁. Optionally, the alkyl groups are substituted by one or more substituents chosen independently from halogen, alkoxy, and haloalkoxy. Examples of suitable alkyl groups include, but are not limited to, —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, 1-hexyl and the like. Preferred alkyl groups are linear or branched, most preferably, linear. Preferred cyclic alkyl groups are cyclopropyl, cyclobutyl, cyclopentyl, and cyclohexyl, most preferably cyclopentyl.

Each instance of acyl, or alkyl individually is optionally unsaturated, and optionally substituted with halogen, alkoxy, and haloalkoxy. In the context of this invention, acyl moieties are alkyl moieties wherein the proximal carbon atom is substituted by an oxo moiety (—O). In the context of the invention, halogen is fluorine (F), chlorine (Cl), bromine (Br), or iodine (I). Preferred halogens for compounds according to the invention are fluorine, chlorine, and bromine, more preferred halogens are chlorine or bromine, a most preferred halogen is chlorine. In the context of this invention, alkoxy is —O-alkyl, preferably wherein said alkyl is a C1-C6 linear or branched alkyl, more preferably a C1-C4 linear or branched alkyl. In the context of this invention, haloalkoxy is an alkoxy, wherein said alkyl comprised in said —O-alkyl is substituted with one or more halogens. A most preferred alkoxyl is methoxy. —C(═O)O(CH₂)₀₋₄H is preferably —C(═O)O(CH₂)₀₋₃H, more preferably —C(═O)O(CH₂)₀₋₁H, most preferably —C(═O)O(CH₃.

Alkyl and acyl groups of the invention are optionally unsaturated. In preferred embodiments, alkyl is not unsaturated. Unsaturated alkyl groups are preferably alkenyl or alkynyl groups. In the context of this invention, unsubstituted alkenyl groups have the general formula CₙH₂ₙ₋₁., and may be linear or branched. Examples of suitable alkenyl groups include, but are not limited to, ethenyl, propenyl, isopropenyl, butenyl, pentenyl and the like. Unsubstituted alkenyl groups may also contain a cyclic moiety, and thus have the concomitant general formula CₙH₂ₙ₋₁. Preferred alkenyl groups are linear or branched, most preferably, linear. Highly preferred unsaturated cyclic alkyl groups are aryl groups, such as phenyl. In the context of this invention, unsubstituted alkynyl groups have the general formula CₙH₂ₙ₋₃ and may be linear or branched. Unsubstituted alkynyl groups may also contain a cyclic moiety, and thus have the concomitant general formula CₙH₂ₙ₋₅. Optionally, the alkynyl groups are substituted by one or more substituents further specified in this document. Examples of suitable alkynyl groups include, but are not limited to, ethynyl, propargyl, n-but-2-ynyl, and n-but-3-ynyl. Preferred alkyl groups are linear or branched, most preferably linear.

In preferred embodiments, C1-6 alkyl when optionally unsaturated and optionally substituted can be C1-6 alkyl, C1-6 acyl, C2-6 alkenyl, C2-6 alkynyl, C3-6 cyclic alkyl, or C5-6 aryl, optionally substituted with one or more moieties selected from halogen, methyl, ethyl, propyl, methoxy, ethoxy, and trifluoromethyl. In preferred embodiments, C1-4 alkyl when optionally unsaturated and optionally substituted can be C1-4 alkyl, C1-4 acyl, C2-4 alkenyl, C2-4 alkynyl, or C3-4 cyclic alkyl, optionally substituted with one or more moieties selected from halogen, methyl, ethyl, propyl, methoxy, ethoxy, and trifluoromethyl.

A leaving group comprised in a compound is defined in the context of this application as a moiety that is attached to the rest of the compound by a covalent bond that can be cleaved heterolytically under mild conditions. Preferred leaving groups are (LG-1), (LG-2), (LG-3), (LG-4), (LG-5), (LG-6), (LG-7), and (LG-8).

A pharmaceutically active substance is defined in the context of this application as a compound or a moiety that is biologically active when it is introduced in a subject, preferably wherein said subject is an animal or a human, more preferably a human. A skilled person understands which pharmaceutically active substances can advantageously be used in this invention. Preferably, the substance has at least one heteroatom through which it is linked to general formula I. Optionally the pharmaceutically active substance can be linked to general formula I via a linker, which is preferably a linker of at most 10 atoms in length, wherein the atoms are preferably selected from C, O, and N, more preferably the atoms are C with at most one O and at most one N, wherein the linker is optionally substituted with one or more O. For instance a preferred linker is —NH—CH₂—CH₂—CH₂—CH₂—CH₂—C(═O)—, also known as aminohexanoic acid. Preferred pharmaceutically active substances are small molecules with a molecular weight of at most 750 Da, more preferably at most 500 Da. Preferred classes of pharmaceutically active substances are antibiotics and chemotherapeutic agents. Preferred chemotherapeutic agents are anthracyclines (aclarubicin, daunorubicin, doxorubicin, epirubicin, idarubicin, amrubicin, pirarubicin, valrubicin, zorubicin), anthracenediones (mitoxantrone, pixantrone), streptomyces (actinomycin, bleomycin, mitomycin, plicamycin), topoisomerase inhibitors: camptotheca (camptothecin, topotecan, irinotecan, rubitecan, belotecan), podophyllum (etoposide, teniposide); tyrosine kinase inhibitors: axitinib, bosutinib, cediranib, dasatinib, erlotinib, gefitinib, imatinib, lapatinib, lestaurtinib, neratinib, nilotinib, semaxanib, sorafenib, sunitinib, vandetanib; cyclin-dependent kinase inhibitors: alvocidib, seliciclib; photosensitizers: aminolevulinic acid/methyl aminolevulinate, efaproxiral, porphyrin derivatives (porfimer sodium, talaporfin, temoporfin, verteporfin) of which anthracyclines are most preferred, and a preferred anthracycline is doxorubicin. Doxorubicin is preferably linked to the remainder of general formula (I) via its nitrogen atom, more preferably while forming a carbamate.

A targeting moiety is preferably an antibody, wherein an antibody refers to polyclonal antibodies, monoclonal antibodies, humanized antibodies, single-chain antibodies, and fragments thereof such as Fab, F(ab)2, Fv, Fab, F(ab)2, scFv, minibody, biabody, scFv-Fc, and other fragments that retain the antigen binding function of the parent antibody. As such, a targeting moiety may preferably refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold. A targeting moiety can also be an aptamer or a lectin.

A small organic molecule preferably has a molecular weight of at most 750 Da, more preferably at most 500 Da. A preferred small organic molecule comprises at least one heteroatom.

Examples of small organic molecules are small fluorophores such as coumarins, preferably aminocoumarins such as 7-aminocoumarins. Preferred coumarins are methylcoumarins such as 4-methylcoumarins. A most preferred coumarin is 7-amino-4-methylcoumarin.

### Conjugates

In a further aspect, the invention provides a conjugate comprising a moiety conjugated to a compound according to the invention. Such a conjugate is called a conjugate according to the invention in the context of this application. The moiety is called the conjugated moiety in this context.

In preferred embodiments, the conjugated moiety is a pharmaceutically active substance, a macromolecule, or a biomolecule, more preferably a protein, most preferably an antibody.

In the context of this application, a macromolecule is preferably defined as a molecule having an molecular mass of at least 1000 Daltons. Preferably the mass is not larger than 100 kDa.

A biomolecule is preferably defined as a molecule obtained or derived from a naturally occurring organism. A protein refers to a polymer of amino acids, which may be optionally substituted (e.g. glycosylated) or modified (e.g. crosslinked). A preferred protein comprises from 10 to 1000 amino acids. A highly preferred protein is an antibody or a fragment thereof, preferably an antibody.

An antibody refers to polyclonal antibodies, monoclonal antibodies, humanized antibodies, single-chain antibodies, and fragments thereof such as Fab, F(ab)2, Fv, Fab, F(ab)2, scFv, minibody, biabody, scFv-Fc, and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold.

### Methods for Producing a Conjugate

In a further aspect, the invention provides a method for producing a conjugate according to the invention, the method comprising the steps of:
i) providing a compound according to the invention;
ii) providing a second compound, preferably wherein said second compound is a small organic molecule, a pharmaceutically active substance, a macromolecule, or a biomolecule, more preferably wherein said second compound is a pharmaceutically active substance, a macromolecule, or a biomolecule, more preferably wherein said second compound is a pharmaceutically active substance of a protein, even more preferably wherein said second compound is a protein, most preferably wherein said second compound is an antibody;
iii) reacting the compound provided in step i) with the second compound to form a conjugate; and optionally
iv) isolating the conjugate obtained in step iii).

### Compositions

In a further aspect, the invention provides a composition comprising at least one compound according to the invention, and a pharmaceutically acceptable excipient. Such a composition is referred to herein as a composition according to the invention. Preferred compositions according to the invention are pharmaceutical compositions. In preferred embodiments, the composition according to the invention is formulated for oral, sublingual, parenteral, intravascular, intravenous, subcutaneous, or transdermal administration, optionally for administration by inhalation; preferably for oral administration. Suitable excipients are water such as water for injection, and pharmaceutically acceptable buffers such as PBS. Further suitable pharmaceutically acceptable excipients include processing agents and delivery modifiers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003), 21^{st} edition (2005) and 22^{nd} edition (2012), and a skilled person can select a suitable one based on the intended application.

In preferred embodiments, a composition according to the invention comprises a tetrazine, more preferably 1,2,4,5-tetrazine, even more preferably a tetrazine of general formula (Tz), wherein Q^{Tz} is in each instance chosen independently from —H, phenyl, 2-pyridyl, 3-pyridyl, 4-pyridyl, 2,6-pyrimidyl, 2,5-pyrimidyl, 3,5-pyrimidyl, and 2,4-pyrimidyl, most preferably wherein Q^{Tz} is in each instance chosen independently from 2-pyridyl, 3-pyridyl, 4-pyridyl. In the context of this application, dipyridyl tetrazine refers to a compound of general formula (Tz), wherein Q^{Tz} is in each instance chosen independently from 2-pyridyl, 3-pyridyl, and 4-pyridyl.

### Medical Use

In a further aspect, the invention provides in a compound according to the invention, a conjugate according to the invention, or a composition according to the invention, for use as a medicament, preferably for use in the treatment or diagnosis of cancer, more preferably treatment.

In a further aspect, the invention provides in vivo, in vitro, or ex vivo method, or a method of treatment, comprising the administration of a compound according to the invention, a conjugate according to the invention, or a composition according to the invention. More preferably, the method of treatment is for the treatment or diagnosis of cancer, more preferably treatment.

Compounds for use as a medicament preferably comprise at least one of a pharmaceutically active substance and a targeting moiety at R^{a}, R^{b}, R^{c}, or R^{d}. Compounds for use as a medicament can also comprise both of a pharmaceutically active substance and a targeting moiety at R^{a}, R^{b}, R^{c}, or R^{d}. Preferably the pharmaceutically active substance is at R^{a} or R^{b}. Preferably the targeting moiety is at R^{c} or R^{d}. When the medicament is for treatment, a pharmaceutically active substance is preferably comprised, more preferably both a pharmaceutically active substance and a targeting moiety are comprised. When the medicament is for diagnosis, a targeting moiety is preferably comprised.

### General Definitions

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

When a structural formula or chemical name is understood by the skilled person to have chiral centers, yet no chirality is indicated, for each chiral center individual reference is made to all three of either the racemic mixture, the pure R enantiomer, or the pure S enantiomer.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 5% of the value.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

### LEGENDS TO THE FIGURES

FIG. 1: The kinetics of TCO E-02a and 3,6-di(pyridin-2-yl)-1,2,4,5-tetrazine with a concentration of 10 and 50 μM at 20° C. in MeCN.

FIG. 2: ¹⁹F-NMR kinetic study of the click-to-release reaction of 20 mM TCO (E-03a) with 3,6-di(pyridin-2-yl)-1,2,4,5-tetrazine at 20° C.

### EXAMPLES

### Example 1—Synthesis of Compounds and Intermediates

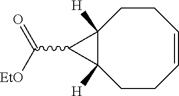 [SMILES: CCOC(=O)C1[C@H]2CC/C=C\CC[C@@H]12]

### Synthesis of ethyl (1R,4Z,8S,9r)-bicyclo[6.1.0]non-4-ene-9-carboxylate (Z-01) and ethyl (1R,4Z,8S,9s)-bicyclo[6.1.0]non-4-ene-9-carboxylate (Z-02)

A 1-I flask was charged with 1,5-cyclooctadiene (466 g, 530 mL, 6 equiv, 4.31 mol) and copper(II) acetylacetonate (14.1 g, 0.075 equiv, 53.9 mmol). The mixture was stirred and purged with argon for 15 minutes. Next, the temperature was elevated to 90° C. (ext) followed by the addition of ethyl diazoacetate (100.00 g, 90.9 mL, 82.0% Wt, 1 equiv, 719 mmol) over ca. 3 hours with a piston pump (flow rate of 0.555 ml/min). A TLC (10% EtOAc in heptane) showed complete consumption of the ethyl diazoacetate 30 minutes after the addition was finished. The mixture was cooled to 40° C. and the flask was placed in a distillation setup with the collection flask place in LN₂. The 1,5-COD was distilled off from the reaction mixture under reduced pressure at 40° C. To the distillation residue was added ammonium hydroxide (25 g, 28 mL, 25% Wt, 0.25 equiv, 180 mmol) in brine (250 ml). The aqueous layers were back extracted with diethyl ether (2×500 ml). Subsequently, the organic layer was washed with ammonium hydroxide (10 g, 11 mL, 25% Wt, 0.10 equiv, 71.9 mmol) in brine (250 ml), an aqueous sat. Na₄EDTA solution (250 ml), dried over MgSO₄ and concentrated to afford the crude product as a yellow oil in 200 g yield with a significant 1,5-COD content. Z-01: ¹H NMR [400 MHz, δ (ppm), CDCl₃]: 5.68-5.59 (m, 2H), 4.10 (q, J=7.2 Hz, 2H), 2.35-2.25 (m, 2H), 2.24-2.14 (m, 2H), 2.13-2.02 (m, 2H), 1.61-1.53 (m, 2H), 1.53-1.42 (m, 2H), 1.25 (t, J=7.2 Hz, 3H), 1.18 (t, J=4.5 Hz, 1H). RF (heptane/EtOAc 24:1): 0.20. Z-02: ¹H NMR [400 MHz, δ (ppm), CDCl₃]: 5.64-5.55 (m, 2H), 4.10 (q, J=7.1 Hz, 2H), 2.55-2.44 (m, 2H), 2.24-2.14 (m, 2H), 2.09-1.98 (m, 2H), 1.86-1.76 (m, 2H), 1.69 (t, J=8.8 Hz, 1H), 1.41-1.32 (m, 2H), 1.25 (t, J=7.1 Hz, 3H). RF (heptane/EtOAc 24:1): 0.30.

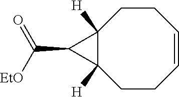 [SMILES: CCOC(=O)[C@H]1[C@@H]2CC/C=C\CC[C@@H]21]

### Synthesis of ethyl (1R,8S,9R, Z)-bicyclo[6.1.0]non-4-ene-9-carboxylate (Z-01

Ethyl (Z)-bicyclo[6.1.0]non-4-ene-9-carboxylate (6.0 g, 1 equiv, 31 mmol) was loaded into a dropping funnel. A dry 100-ml reaction vial was purged with argon for 15 minutes and charged with a 1M KOtBu (4.5 g, 40 mL, 1.00 molar, 1.30 equiv, 40 mmol) solution in THF and dry ethanol (1.6 g, 2.0 mL, 1.1 equiv, 34 mmol). The substrate solution was drop wise added to the stirred ethanol-KOtBu solution at r.T. under argon atmosphere. After full conversion was observed on TLC, the reaction was quenched with ethanol (4 ml) and a large portion of THF (ca. 20 ml) was removed via distillation. After ethanol (40 ml) was added, the reaction mixture was refluxed overnight. EtOAc (200 ml), brine (150 ml) and 1M aqueous HCl (45 ml) were added to the reaction mixture. After the organic layer was collected, the aqueous layer was back extracted with EtOAc (100 ml). The combined organic layers were then washed with 1M aqueous NaOH (150 ml) followed by brine (150 ml). After the collected organic layer was dried over MgSO₄, the mixture was concentrated to afford the crude product in 5.2 g (87% of theory) yield. The mixture was purified via distillation under reduced pressure at 150° C. Z-01: ¹H NMR [400 MHz, δ (ppm), CDCl₃]: 5.68-5.59 (m, 2H), 4.10 (q, J=7.2 Hz, 2H), 2.35-2.25 (m, 2H), 2.24-2.14 (m, 2H), 2.13-2.02 (m, 2H), 1.61-1.53 (m, 2H), 1.53-1.42 (m, 2H), 1.25 (t, J=7.2 Hz, 3H), 1.18 (t, J=4.5 Hz, 1H). RF (heptane/EtOAc 24:1): 0.20.

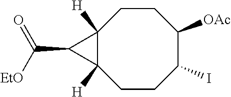 [SMILES: CCOC(=O)[C@H]1[C@@H]2CC[C@@H](OC(C)=O)[C@H](I)CC[C@@H]21]

### Synthesis of ethyl (1S,8R,9R)-4-acetoxy-5-iodobicyclo[6.1.0]nonane-9-carboxylate (Z-03

A suspension of ethyl (Z)-bicyclo[6.1.0]non-4-ene-9-carboxylate (20.4 g, 1 equiv, 105 mmol) and sodium acetate (25.8 g, 3.0 equiv, 315 mmol) in acetic acid (252 g, 240 mL, 40 equiv, 4.20 mol) was stirred and cooled with a water bath until the temperature stabilized to ambient temperature. Next, NIS (28.4 g, 1.2 equiv, 126 mmol) was added and the mixture was stirred at ambient temperature for 3 hours. After full conversion was observed on TLC, brine (50 ml) and heptane (200 ml) were added to the reaction mixture. The organic layer was collected and washed with brine (250 ml), a mixture of sat. aqueous sodium thiosulfate solution (50 ml) and brine (200 ml) followed by sat. aqueous sodium bicarbonate solution (200 ml). The aqueous layers were back-extracted in similar order with two additional portions of heptane (2×200). The combined organic layers were dried over MgSO₄ and concentrated to afford the crude product in 39 g. The crude was purified over silica (2×120 g) with gradient of 25% to 75% EtOAc in heptane to afford the purified product as white crystals in 33.2 g (83% of theory) yield. ¹H NMR (400 MHz, CDCl₃) δ 4.86 (ddd, J=7.7, 6.6, 3.5 Hz, 1H), 4.75 (td, J=7.8, 5.3 Hz, 1H), 4.08 (q, J=7.2 Hz, 2H), 2.24-2.17 (m, 2H), 2.15-2.08 (m, 1H), 2.07 (s, 3H), 2.05-1.96 (m, 2H), 1.84-1.70 (m, 1H), 1.54-1.46 (m, 1H), 1.46-1.38 (m, 1H), 1.34-1.25 (m, 2H), 1.23 (t, J=7.1 Hz, 3H), 1.15 (t, J=4.3 Hz, 1H). ¹³C NMR (101 MHz, CDCl₃) δ 173.66, 169.75, 73.30, 60.44, 37.53, 33.71, 33.22, 26.19, 26.19, 26.04, 25.71, 23.61, 21.49, 14.29. RF (heptane/EtOAc 3:1) 0.14.

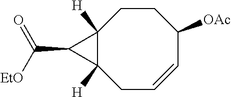 [SMILES: CCOC(=O)[C@H]1[C@@H]2CC[C@@H](OC(C)=O)/C=C\C[C@@H]21]

### Synthesis of ethyl (1S,5R,8R,9S, Z)-5-acetoxybicyclo[6.1.0]non-3-ene-9-carboxylate (Z-04

To a stirred solution of ethyl 4-acetoxy-5-iodobicyclo[6.1.0]nonane-9-carboxylate (33.2 g, 1 equiv, 87.3 mmol) in toluene (241 g, 279 mL, 30 equiv, 2.62 mol) was added DBU (39.9 g, 39.5 mL, 3.0 equiv, 262 mmol). The mixture was stirred at 100° C. (ext.) overnight. The reaction mixture was cooled with an ice bath and filtered to remove solids. The filter residue was washed with toluene (2×50 ml). The filtrate was washed with brine (2×250 ml) and the aqueous layers were back extracted with toluene (100 ml). The combined organic layers were dried over MgSO₄ and concentrated to afford a yellow oil as the crude product in 25 g. The crude was purified over silica (2×120 g) with a gradient of 10 to 20% EtOAc in heptane to afford the purified product as white crystals in 19.6 g (89% of theory) yield. ¹H NMR (500 MHz, CDCl₃) δ 5.81 (dddd, J=11.3, 9.2, 6.9, 2.0 Hz, 1H), 5.62 (dd, J=11.1, 5.3 Hz, 1H), 5.32 (dd, J=10.6, 5.2 Hz, 1H), 4.10 (q, J=7.2 Hz, 2H), 2.44-2.34 (m, 2H), 2.05 (s, 3H), 1.91-1.85 (m, 1H), 1.79-1.63 (m, 3H), 1.44-1.28 (m, 3H), 1.25 (t, J=7.1 Hz, 3H). ¹³C NMR (126 MHz, CDCl₃) δ 173.50, 170.19, 133.93, 129.18, 75.30, 60.37, 36.46, 31.22, 28.67, 26.92, 26.43, 25.42, 21.31, 14.25. RF (heptane/EtOAc 4:1) 0.26.

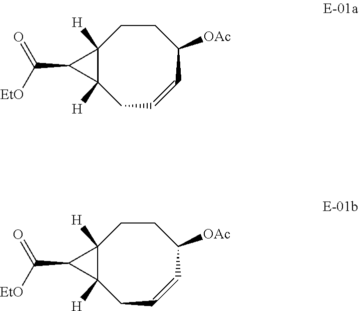

### Synthesis of ethyl (1S,5R,8R,9S, E)-5-acetoxybicyclo[6.1.0]non-3-ene-9-carboxylate (E-01a. E-01b

A custom-made long-necked flask was charged with an aqueous solution of silver nitrate (7.00 g, 2.97 equiv, 41.2 H mmol) in water (100 ml). Next, a solution of ethyl (1R,5S,8S,9R, Z)-5-acetoxybicyclo[6.1.0]non-3-ene-9-carboxylate (3.50 g, 92.5 mL, 150H mmolar, 1 equiv, 13.9 mmol) and methyl benzoate (3.84 g, 3.56 mL, 2.03 equiv, 28.2 mmol) in deoxygenated heptane (160 ml) and MTBE (40 ml) HE-b was loaded into a UV irradiation setup (as described in Blanco-Ania et al., ChemPhotoChem 2018, 2(10), 898-905.). The continuous process ran overnight for 16 hours. Next, the biphasic reaction mixture was loaded into a separation funnel and the aqueous layer was collected. The organic layer was washed with water (100 ml). The combined aqueous layers were washed with heptane (100 ml). Subsequently, 25% aqueous ammonium hydroxide (25 ml) was added to the aqueous layer before extracting it with EtOAc (100 ml). The combined organic layers were dried over MgSO₄ and concentrated to afford a diastereomeric mixture with a ratio of 7:13 (E-01a:E-01b) of two products as clear oil in 1.6 g yield. (E-01a) ¹H NMR (500 MHz, CDCl₃) δ 6.11 (ddd, J=16.8, 10.9, 5.9 Hz, 0.35H), 5.55 (dd, J=16.9, 3.4 Hz, 0.35H), 5.15 (q, J=3.1 Hz, 0.35H), 4.14-4.05 (m, 0.70H), 2.67 (dt, J=12.6, 6.0 Hz, 0.35H), 2.51-2.45 (m, 0.35H), 2.25-2.20 (m, 0.35H), 2.08 (m, 0.35H), 2.05 (q, 0.70H), 2.00-1.95 (m, 0.35H), 1.80-1.75 (m, 0.35H), 1.60-1.55 (m, 0.70H), 1.55-1.45 (m, 0.35H), 1.24 (dt, J=10.5, 7.1 Hz, 1.05H). ¹³C NMR (126 MHz, CDCl₃) δ 174.95, 170.04, 131.77, 129.56, 70.78, 60.57, 37.26, 31.37, 29.29, 26.75, 21.17, 20.62, 20.62, 14.29. RF (EtOAc/heptane 1:1) 0.54. (E-01b) ¹H NMR (500 MHz, CDCl₃) δ 5.91 (ddd, J=16.0, 9.1, 6.4 Hz, 0.65H), 5.67 (ddd, J=16.6, 9.7, 1.5 Hz, 0.65H), 5.01 (td, J=9.8, 5.3 Hz, 0.65H), 4.14-4.05 (m, 1.30H), 2.86 (dtt, J=14.7, 9.2, 1.1 Hz, 0.65H), 2.40-2.35 (m, 0.65H), 2.38-2.30 (m, 0.65H), 2.17 (m, 0.65H), 1.55-1.45 (m, 0.65H), 1.40 (t, J=5.6 Hz, 0.65H), 1.25 (dt, J=10.5, 7.1 Hz, 3H), 1.25-1.20 (m, 0.65H), 0.77 (dt, J=15.4, 11.4 Hz, 0.65H). ¹³C NMR (126 MHz, CDCl₃) δ 174.28, 170.62, 136.17, 131.20, 77.46, 60.47, 37.67, 36.47, 34.61, 30.23, 29.06, 28.47, 21.25, 14.26. RF (EtOAc/heptane 1:1) 0.54.

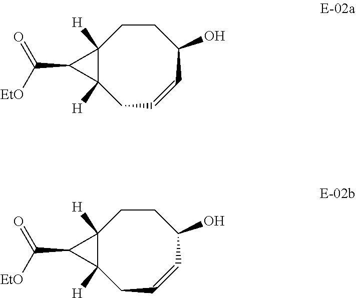

### Synthesis of ethyl (1S,5R,8R,9S, E)-5-hydroxybicyclo[6.1.0]non-3-ene-9-H carboxylate (E-02a. E-02b

A solution of ethyl (1R,5S,8S,9R, E)-5-acetoxybicyclo[6.1.0]non-3-ene-9-carboxylate (2.00 g, 1 equiv, 7.93 mmol) and potassium carbonate (2.19 g, 2.0 equiv, 15.9 mmol) in ethanol (15.7 g, 19.9 mL, 43 equiv, 341 mmol) was stirred at ambient temperature overnight. The flask was shielded from light with aluminium foil. After completion was observed with TLC, acetic acid (2.00 g, 1.91 mL, 4.2 equiv, 33.3 mmol) in a 1:1 water-brine mixture (50 ml) was added to quench the reaction. After EtOAc (25 ml) was added and separated from the aqueous layer, the organic layer was washed with aqueous sat. NaHCO₃ (25 ml), brine (25 ml). All aqueous layers were back-extracted with EtOAc (2×25 ml). The combined organic layers were dried over MgSO₄ and concentrated to afford the crude product as a yellow oil in 1.8 g yield. The diastereomers were separated over silica (80 g, 10% acetone in toluene) to afford E-02a in 470 mg yield and E-02b in 980 mg yield. (E-02a) ¹H NMR (500 MHz, CDCl₃) δ 6.26 (ddd, J=16.8, 10.9, 5.9 Hz, 1H), 5.55 (ddd, J=16.8, 3.1, 0.9 Hz, 1H), 4.36 (q, J=3.0 Hz, 1H), 4.08 (qd, J=7.1, 2.1 Hz, 2H), 2.66 (dt, J=12.5, 6.0 Hz, 1H), 2.49-2.42 (m, 1H), 2.25 (dd, J=16.1, 11.9 Hz, 1H), 2.00-1.91 (m, 1H), 1.87 (q, J=8.0 Hz, 1H), 1.79-1.74 (m, 1H), 1.68 (t, J=13.1 Hz, 1H), 1.55-1.48 (m, 2H), 1.46-1.40 (m, 1H), 1.24 (t, J=7.2 Hz, 3H). ¹³C NMR (126 MHz, CDCl₃) δ 175.25, 133.55, 130.42, 68.52, 60.49, 39.75, 31.73, 29.13, 27.05, 20.57, 19.31, 14.27. RF (EtOAc/heptane 1:1) 0.37 (E-02b) ¹H NMR (500 MHz, CDCl₃) δ 5.76 (ddd, J=15.8, 8.9, 6.5 Hz, 1H), 5.62 (ddd, J=16.5, 9.4, 1.4 Hz, 1H), 4.08 (dd, J=9.8, 5.2 Hz, 1H), 4.05 (q, J=7.1 Hz, 2H), 2.81 (dtt, J=14.8, 9.3, 1.2 Hz, 1H), 2.34-2.29 (m, 1H), 2.28-2.23 (m, 1H), 2.16-2.08 (m, 1H), 2.10-2.05 (m, 1H), 1.43 (dddd, J=13.1, 11.4, 9.7, 0.8 Hz, 1H), 1.32-1.29 (m, 1H), 1.20 (t, J=7.1 Hz, 3H), 1.18-1.13 (m, 1H), 0.68 (dt, J=15.4, 11.3 Hz, 1H). ¹³C NMR (126 MHz, CDCl₃) δ 174.53, 140.34, 129.39, 76.10, 60.44, 38.02, 37.75, 30.33, 29.31, 28.63, 28.50, 14.23. RF (EtOAc/heptane 1:1) 0.27.

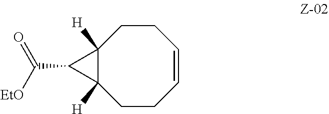 [SMILES: CCOC(=O)[C@@H]1[C@@H]2CC/C=C\CC[C@@H]21]

### Ethyl (1R,4Z,8S,9s)-bicyclo[6.1.0]Non-4-Ene-9-carboxylate (Z-02

Z-02 was separated from Z-01 by subjecting a mixture of Z-01 and Z-02 to silica gel column chromatography.

### Ethyl (1S,4R,5R,8R,9S)-4-acetoxy-5-iodobicyclo[6.1.0]nonane-9-carboxylate (Z-021

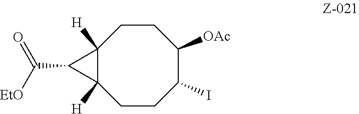 [SMILES: CCOC(=O)[C@@H]1[C@@H]2CC[C@@H](OC(C)=O)[C@H](I)CC[C@@H]21]

Ethyl (1R,8S,9s, Z)-bicyclo[6.1.0]non-4-ene-9-carboxylate (Z-02, 501 mg, 2.6 mmol) and NIS (696 mg, 3.1 mmol) were dissolved in acetic acid (3.0 mL) under an inert atmosphere. To the solution was added 3.0 mL of a saturated NaOAc in AcOH solution. The reaction mixture was stirred for 72 h. after which it was diluted with ethyl acetate (20 mL), water (15 mL), brine (15 mL) and 10% aqueous Na₂S₂O₃ solution (10 mL). The organic phase was separated from the aqueous layers and the aqueous layers were extracted with ethyl acetate (5×40 mL). The combined organic layers were dried with MgSO₄, concentrated in vacuo and purified with silica gel column chromatography (0%→20% EtOAc in n-heptane) to afford Z-021 (800 mg, 82%) as a yellow dense liquid. TLC (EtOAc/n-heptane, 3:7 v/v): R_{f}=0.55. ¹H NMR (500 MHz, CDCl₃) δ 4.95 (ddd, J=7.8, 6.8, 3.2 Hz, 1H), 4.76 (ddd, J=9.3, 7.8, 3.8 Hz, 1H), 4.14 (q, J=7.1 Hz, 2H), 2.40-2.23 (m, 2H), 2.22-2.08 (m, 6H), 1.91-1.64 (m, 4H), 1.42 (dtd, J=10.9, 8.7, 4.0 Hz, 1H), 1.35-1.21 (m, 4H). ¹³C NMR (126 MHz, CDCl₃) δ 171.8, 169.8, 74.0, 60.2, 38.2, 33.9, 33.9, 23.6, 23.5, 21.7, 21.3, 21.3, 18.7, 14.5.

### Ethyl (1R,5R,8S,9S, Z)-5-acetoxybicyclo[6.1.0]Non-3-Ene-9-carboxylate(Z-022

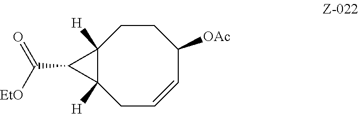 [SMILES: CCOC(=O)[C@@H]1[C@@H]2CC[C@@H](OC(C)=O)/C=C\C[C@@H]21]

Ethyl (1S,4R,5R,8R,9S)-4-acetoxy-5-iodobicyclo[6.1.0]nonane-9-carboxylate (Z-021 800 mg, 2.1 mmol) was dissolved in dry toluene (10 mL). To the solution was added DBU (1 mL, 6.3 mmol) and the solution was heated to 100° C. The reaction was stirred for a total of 2 days at 100° C. The reaction was cooled to room temperature, washed with water (10 mL), 1M HCl (10 mL) and brine (10 mL). Subsequently the combined aqueous layers were extracted with toluene (10 mL) and the combined organic layers were dried with MgSO₄, concentrated in vacuo and purified with silica gel column chromatography (0%→30% EtOAc in n-heptane) to afford Z-022 (254 mg, 48%) as a colorless oil. TLC (EtOAc/n-heptane, 3:7 v/v): R_{f}=0.66. ¹H NMR (500 MHz, CDCl₃) δ 5.82 (dddd, J=11.1, 9.8, 7.0, 2.1 Hz, 1H), 5.57 (ddd, J=10.9, 5.2, 1.2 Hz, 1H), 5.48-5.39 (m, 1H), 4.12 (q, J=7.1 Hz, 2H), 2.60 (dddd, J=13.7, 12.7, 9.8, 1.3 Hz, 1H), 2.29-2.14 (m, 1H), 2.11-2.02 (m, 4H), 1.98-1.90 (m, 2H), 1.76-1.64 (m, 2H), 1.45 (dtd, J=12.2, 8.7, 3.3 Hz, 1H), 1.34-1.22 (m, 4H). ¹³C NMR (126 MHz, CDCl₃) δ 172.0, 170.2, 134.0, 130.2, 75.6, 60.1, 36.6, 28.5, 23.3, 22.2, 21.5, 20.6, 14.5. HRMS (m/z): [M+Na]+ calcd. for C₁₄H₂₀O₄Na: 275.1259, found 275.1243.

### Ethyl (1R,8S,9S, E)-5-acetoxybicyclo[6.1.0]Non-3-Ene-9-carboxylate (E-021a/E-021b

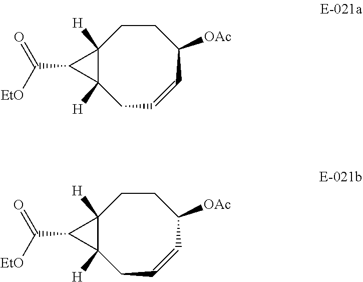

A custom-made long-necked flask was charged with an aqueous solution of silver nitrate (2.69 g, 15.85 mmol) in water (3 ml). Next, a solution of ethyl (1R,5R,8S,9S, Z)-5-acetoxybicyclo[6.1.0]non-3-ene-9-carboxylate (Z-022, 2.00 g, 40 mmolar, 7.93 mmol) and methyl benzoate (400 μL, 3.17 mmol) in deoxygenated heptane (20 ml) was loaded into a UV irradiation setup (as described in Blanco-Ania et al., ChemPhotoChem 2018, 2 (10), 898-905.). The continuous process ran for 43 h. The extraction vial was disconnected from the setup and residual solution in the photoreactor was collected by flushing the remaining system with heptane (150 ml). Next, the biphasic reaction mixture was loaded into a separation funnel and additional water (150 ml) was added before starting the extraction. After phase separation, the organic layer was washed with a solution of silver nitrate (1.5 g) in water (100 ml). The combined aqueous layers were then back extracted with heptane (100 mL) to remove residual starting material from the aqueous layers. Next, ammonium hydroxide (5.6 mL, 25% Wt, 35.67 mmol) was added to the aqueous layer to decomplex the product from the silver ions. The turbid aqueous solution was extracted with ethyl acetate (3×200 ml). The combined organic layers were washed with water (100 ml), dried with MgSO₄ and concentrated to afford a diastereomeric mixture with a ratio of 1:1 (E-021a:E-021b) of two products as clear oil (610 mg, 31%). HRMS (m/z): [M+Na]+ calcd. for C₁₄H₂₀O₄Na: 275.1259, found 275.1241.

E-021a (Axial): TLC (EtOAc/n-heptane, 1:1 v/v): R_{f}=0.73. ¹H NMR (500 MHz, CDCl₃) δ 5.86-5.80 (m, 2H), 5.06-5.00 (m, 1H), 4.16-4.09 (m, 2H), 2.68-2.60 (m, 1H), 2.51 (ddd, J=11.7, 9.2, 1.8 Hz, 1H), 2.48-2.43 (m, 1H), 2.07 (s, 3H), 1.81 (t, J=9.0 Hz, 1H), 1.74-1.71 (m, 2H), 1.62-1.59 (m, 1H), 1.58-1.54 (m, 1H), 1.29-1.25 (m, 3H), 1.25-1.22 (m, 1H). ¹³C NMR (126 MHz, CDCl₃) δ 171.7, 170.7, 134.0, 131.3, 69.9, 60.1, 38.8, 26.1, 26.0, 24.2, 22.0, 21.4, 17.4, 14.5.
E-021b (Equatorial): TLC (EtOAc/n-heptane, 1:1 v/v): R_{f}=0.73. ¹H NMR (500 MHz, CDCl₃) δ 6.32 (dddd, J=16.8, 8.4, 6.8, 0.8 Hz, 1H), 5.62 (ddd, J=16.8, 9.7, 1.5 Hz, 1H), 5.15-5.07 (m, 1H), 4.16-4.09 (m, 2H), 2.79-2.71 (m, 1H), 2.34 (ddd, J=14.5, 6.7, 2.1 Hz, 1H), 2.23 (dt, J=12.0, 6.2 Hz, 1H), 2.10-2.08 (m, 1H), 2.05 (s, 3H), 1.90-1.84 (m, 1H), 1.73 (d, J=1.4 Hz, 1H), 1.50-1.41 (m, 2H), 1.27 (t, J=0.9 Hz, 3H), 1.15-1.07 (m, 1H). ¹³C NMR (126 MHz, CDCl₃) δ 171.7, 170.7, 135.3, 134.3, 77.9, 60.1, 35.0, 34.8, 26.1, 25.4, 24.0, 24.0, 21.4, 14.5.

### Synthesis of ethyl (1S,5R,8R,9S, E)-5-(((perfluorophenoxy)carbonyl)oxy)bicyclo[6.1.0]non-3-ene-9-carboxylate (E-03a

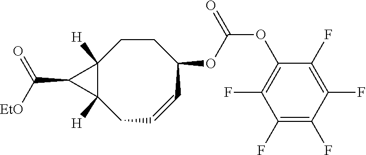

To a stirred solution of ethyl (1R,5S,8S,9R, E)-5-hydroxybicyclo[6.1.0]non-3-ene-9-carboxylate (E-02a, 38.0 mg, 1 equiv, 181 μmol) and DIPEA (70.1 mg, 94.4 μL, 3.0 equiv, 542 μmol) in DCM (1.30 g, 988 μL, 85 equiv, 15.4 mmol) was added DPFPC (196 mg, 2.75 equiv, 497 μmol) at 0° C. After the addition was complete, the mixture was covered from light and allowed to warm up to ambient temperature. After completion, water (1 ml) was added and subsequently the mixture was neutralized with acidic acid. The organic layer was washed with water (2 ml). The collected organic layer was dried over MgSO₄ and concentrated to afford crude product in 190 mg yield. Purification over silica (gradient of 10 to 500/EtOAc in heptane, 4 g silica) afforded an isolated yield of 24 mg (320/of theory). ¹H NMR (400 MHz, CDCl₃) δ 6.27 (ddd, J=16.9, 10.9, 5.9 Hz, 1H), 5.57 (dd, J=16.9, 3.3 Hz, 1H), 5.17 (d, J=3.1 Hz, 1H), 4.13 (q, J=7.2 Hz, 2H), 2.76 (dt, J=12.8, 6.3 Hz, 1H), 2.57-2.47 (m, 1H), 2.34-2.19 (m, 2H), 2.08 (s, 1H), 1.88 (t, J=13.9 Hz, 1H), 1.64-1.54 (m, 2H), 1.53-1.46 (m, 1H), 1.26 (t, J=7.1 Hz, 3H). ¹³C NMR (101 MHz, CDCl₃) δ 174.70, 150.31, 142.46, 141.01, 140.11, 139.12, 138.49, 136.65, 133.26, 127.54, 77.10, 60.66, 37.31, 31.25, 29.41, 26.42, 20.47, 14.19. ¹⁹F NMR (377 MHz, CDCl₃) δ −153.22 (d, J=17.0 Hz, 2F), −157.50 (t, J=21.7 Hz, 1F), −162.04 (dd, J=21.8, 17.0 Hz, 2F). RF (EtOAc/heptane 1:1) 0.55.

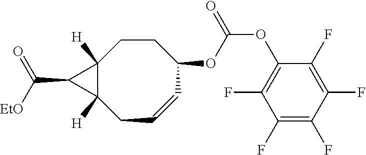

### Synthesis of ethyl (1S,5R,8R,9S, E)-5-(((perfluorophenoxy)carbonyl)oxy)bicyclo[6.1.0]non-3-ene-9-carboxylate (E-03b

To a stirred solution of ethyl (1R,5S,8S,9R, E)-5-hydroxybicyclo[6.1.0]non-3-ene-9-carboxylate (E-02b, 52.0 mg, 1 equiv, 247 μmol) and DIPEA (95.9 mg, 129 μL, 3.0 equiv, 742 μmol) in DCM (1.79 g, 1.35 mL, 85 equiv, 21.0 mmol) was added DPFPC (268 mg, 2.75 equiv, 680 μmol) at 0° C. After the addition was complete, the mixture was covered from light and allowed to warm up to ambient temperature. After completion, water (1 ml) was added and subsequently the mixture was neutralized with acidic acid. The organic layer was washed with water (2 ml). The collected organic layer was dried over MgSO₄ and concentrated to afford the crude product in 270 mg yield. Purification over silica (gradient of 10 to 50% EtOAc in heptane, 4 g silica) afforded an isolated yield of 45 mg (43% of theory). ¹H NMR (400 MHz, CDCl₃) δ 6.01 (ddd, J=16.0, 9.0, 6.5 Hz, 1H), 5.79 (ddd, J=16.6, 9.6, 1.4 Hz, 1H), 5.03 (td, J=9.9, 5.3 Hz, 1H), 4.10 (q, J=7.1 Hz, 2H), 2.99-2.86 (m, 1H), 2.49-2.31 (m, 3H), 2.26-2.18 (m, 1H), 1.70 (dt, J=12.9, 10.7 Hz, 1H), 1.39 (t, J=5.6 Hz, 1H), 1.30-1.19 (m, 1H), 1.25 (t, J=7.1 Hz, 3H), 0.78 (dt, J=15.1, 11.3 Hz, 1H). ¹³C NMR (101 MHz, CDCl₃) δ 174.18, 150.62, 142.57, 140.97, 140.14, 139.16, 138.45, 136.41, 134.56, 132.97, 83.57, 60.63, 37.50, 34.32, 30.06, 28.79, 28.72, 28.49, 14.22. ¹⁹F NMR (377 MHz, CDCl₃) δ −153.16 (d, J=16.9 Hz), −157.66 (t, J=21.7 Hz), −162.15 (dd, J=21.7, 17.0 Hz). RF (EtOAc/heptane 1:1) 0.57.

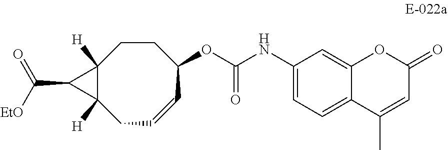

### COOEt-Exo-Axial-TCO-AMC (E-022a

7-Amino-4-methylcoumarin (121 mg, 0.69 mmol) was dissolved in dry toluene (15 mL) and DIPEA (442 μL, 2.54 mmol) and triphosgene (205 mg, 0.69 mmol) were added. The reaction mixture was stirred for 1 h. at 120° C. and allowed to cool down to rt. COOEt-R-TCO-OH (E-02a, 116 mg, 0.52 mmol) was dissolved in dry DCM (10 mL) and DMAP (202 mg, 1.66 mmol) was added. The cooled down 7-amino-4-methylcoumarin solution was added dropwise to the TCO mixture in DCM on ice. The reaction mixture was stirred for 18 h. in the dark. The reaction mixture was concentrated and suspended in EtOAc (80 mL). 1 M aqueous HCl (40 mL) was added and the aqueous phase was separated. The organic layer was washed brine (30 mL). The combined aqueous layers were back extracted with EtOAc (2×20 mL). The combined organic layers were dried with MgSO₄, concentrated in vacuo and purified with silica gel column chromatography (5%→50% EtOAc in n-pentane) to afford E-022a (142.3 mg, 63%) as an off-white solid. TLC (EtOAc/n-pentane, 1:1 v/v): R_{f}=0.54. ¹H NMR (500 MHz, CDCl₃) δ 7.55 (d, J=8.6 Hz, 1H), 7.48-7.43 (m, 2H), 7.15 (s, 1H), 6.25-6.16 (m, 1H), 5.63 (dd, J=16.9, 3.3 Hz, 1H), 5.23 (d, J=3.2 Hz, 1H), 4.17-4.11 (m, 2H), 2.72 (dt, J=12.5, 6.0 Hz, 1H), 2.55-2.48 (m, 1H), 2.43 (d, J=1.3 Hz, 3H), 2.30-2.14 (m, 2H), 2.04 (q, J=6.5 Hz, 1H), 1.86 (t, J=13.6 Hz, 1H), 1.60 (q, J=8.7 Hz, 3H), 1.50 (dd, J=9.1, 6.4 Hz, 1H), 1.30 (t, J=7.1 Hz, 3H). ¹³C NMR (126 MHz, CDCl₃) δ 175.0, 161.2, 154.5, 152.4, 152.1, 141.6, 132.4, 129.4, 125.5, 115.6, 114.5, 113.3, 106.0, 72.2, 60.7, 37.6, 31.5, 29.5, 26.8, 21.1, 20.8, 18.7, 14.4. HRMS (m/z): [M+Na]+ calcd. for C₂₃H₂₅N₁O₆Na: 434.1579, found 434.1575.

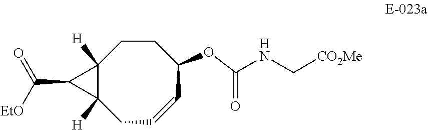

### COOEt-Exo-Axial-TCO-Glycine-OMe (E-023a

To a stirred H solution of ethyl(1R,5S,8S,9R, E)-5-hydroxybicyclo[6.1.0]non-3-ene-9-carboxylate (E-02a, 46 mg, 219 μmol) in dry acetonitrile (3 mL) at 0° C., was added DPFPC (172 mg, 438 μmol), DIPEA (191 μL, 1.09 mmol), and DMAP (2.7 mg, 21.9 μmol). After the addition was complete, the mixture was shielded from light and allowed to warm up to ambient temperature overnight. After completion, the reaction mixture was diluted with diethyl ether (10 mL) and washed with water (2×5 mL). The organic layer was dried with MgSO₄ and concentrated in vacuo. Without further purification, the product was dissolved in dry acetonitrile (2.5 mL) and to this a solution of glycine methyl ester hydrochloride (55 mg, 438 μmol) and DIPEA (95.4 μL, 547 μmol) in dry acetonitrile (2.5 mL) was added. The reaction was shielded from light. Upon completion, the reaction was diluted with EtOAc (10 mL) and extracted with water (2×5 mL). The organic layers were dried with MgSO₄, concentrated in vacuo and purified with silica gel column chromatography (5% acetone in toluene) to afford E-023a (19 mg, 27%) as a slightly yellow oil. TLC (acetone/toluene, 1:9 v/v): R_{f}=0.21. ¹H NMR (500 MHz, CDCl₃) δ 6.13 (ddd, J=16.9, 11.0, 5.9, 1H), 5.56 (dd, J=16.9, 3.3, 1H), 5.22 (t, J=5.6, 1H), 5.08 (q, J=3.1, 1H), 4.11 (qd, J=7.2, 2.6, 2H), 3.97 (dd, J=5.6, 3.1, 2H), 3.76 (s, 3H), 2.67 (dt, J=9.8, 6.0, 1H), 2.46 (t, J=11.9, 1 H), 2.24-2.17 (m, 1H), 2.10 (ddd, J=15.2, 6.7, 2.8, 1H), 2.01-1.89 (m, 1H), 1.76 (t, J=13.8, 1H), 1.65-1.50 (m, 2H), 1.48-1.41 (m, 1H), 1.26 (t, J=7.1, 3H). ¹³C NMR (126 MHz, CDCl₃) δ 175.1, 170.6, 155.5, 131.8, 129.9, 71.8, 60.7, 52.5, 42.7, 31.6, 29.4, 27.1, 20.6, 14.4. HRMS (m/z): [M+Na]+ calcd. for C₁₆H₂₃NO₆Na: 348.1423, found 348.1413.

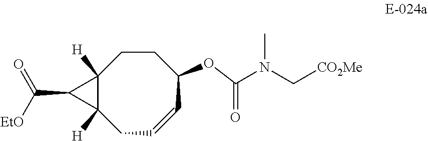

### COOEt-Exo-Axial-TCO-Sarcosine-OMe (E-024a

To a stirred solution of ethyl(1R,5S,8S,9R, E)-5-hydroxybicyclo[6.1.0]non-3-ene-9-carboxylate (E-02a, 50.3 mg, 239 μmol) in dry acetonitrile (3 mL) at 0° C., was added DPFPC (191 mg, 485 μmol), DIPEA (208 μL, 1.2 mmol), and DMAP (3.6 mg, 29 μmol). After the addition was complete, the mixture was shielded from light and allowed to warm up to ambient temperature overnight. After completion, the reaction mixture was diluted with diethyl ether (10 mL) and washed with water (2×5 mL). The organic layer was dried with MgSO₄ and concentrated in vacuo. Without further purification, the product was dissolved in dry acetonitrile (2.5 mL) and to this a solution of sarcosine methyl ester hydrochloride (66.4 mg, 476 μmol) and DIPEA (104 μL, 595 μmol) in dry acetonitrile (2.5 mL) was added. The reaction was shielded from light. Upon completion, the reaction mixture was diluted with EtOAc (10 mL) and it was washed with water (2×5 mL). The organic layer was dried with MgSO₄, concentrated in vacuo and purified with silica gel column chromatography (5% acetone in toluene) to afford E-024a as a colorless oil (31 mg, 31%). TLC (acetone/toluene, 1:9 v/v) R_{f}=0.32. ¹H NMR (500 MHz, CDCl₃) rotamer 1: δ 6.12 (ddd, J=16.8, 10.9, 5.9, 1H), 5.56 (td, J-16.7, 3.3, 1H), 5.11 (dd, J=5.9, 3.0, 1H), 4.11 (dtd, J=10.1, 7.1, 2.8, 2H), 4.06-3.95 (m, 2H), 3.73 (s, 3H), 3.03 (s, 3H), 2.67 (tt, J=12.4, 6.2, 1H), 2.52-2.42 (m, 1H) 2.22 (dd, J=16.2, 11.8, 1H), 2.15-2.10 (m, 1H), 2.10-2.02 (m, 1H), 1.81-1.68 (m, 1H), 1.55 (td, J=11.3, 10.4, 5.8, 2H), 1.45 (ddd, J=15.0, 9.9, 6.1, 1H), 1.26 (td, J=7.1, 2.1, 3H), rotamer 2: δ 5.99 (ddd, J=16.8, 10.9, 5.9, 1H), 5.56 (td, J=16.7, 3.3, 1H), 5.11 (dd, J=5.9, 3.0, 1H), 4.11 (dtd, J=10.1, 7.1, 2.8, 2H), 4.06-3.95 (m, 2H), 3.77 (s, 3H), 2.99 (s, 3H), 2.67 (tt, J=12.4, 6.2, 1H), 2.52-2.42 (m, 1H) 2.22 (dd, J=16.2, 11.8, 1H), 2.15-2.10 (m, 1H), 2.10-2.02 (m, 1H), 1.81-1.68 (m, 1H), 1.55 (td, J=11.3, 10.4, 5.8, 2H), 1.45 (ddd, J=15.0, 9.9, 6.1, 1H), 1.26 (td, J=7.1, 2.1, 3H)¹³C NMR (126 MHz, CDCl₃) rotamer 1: δ 175.1, 170.1, 155.9, 131.5, 130.1, 72.3, 60.7, 52.2, 50.6, 37.6, 35.3, 31.6, 29.4, 26.9, 20.7, 14.4, rotamer 2: δ 175.2, 170.2, 155.2, 131.6, 129.9, 72.2, 60.7, 52.3, 50.7, 37.6, 36.2, 31.6, 29.4, 26.9, 20.6, 14.43. HRMS (m/z): [M+Na]+ calcd. for C₁₇H₂₅NO₆Na: 362.1579, found 362.1598.

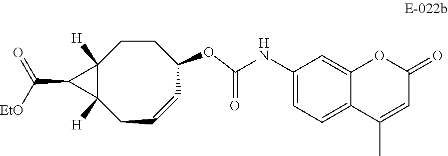

### COOEt-Exo-Equatorial-TCO-AMC (E-022b

7-Amino-4-methylcoumarin (15 mg, 86 μmol) was dissolved in dry toluene (2 mL) and DIPEA (55 μL, 317 μmol) and triphosgene (25 mg, 86 μmol) were added. The reaction mixture was stirred for 1 h. at 120° C. and allowed to cool down to rt. COOEt-NR-TCO-OH (E-02b, 14 mg, 67 μmol) was dissolved in dry DCM (5 mL) and DMAP (26 mg, 214 μmol) was added. The cooled down 7-amino-4-methylcoumarin solution was added dropwise to the TCO mixture in DCM on ice. The reaction mixture was stirred for 18 h. in the dark. The reaction mixture was concentrated in vacuo and purified with silica gel column chromatography (10%→60% EtOAc in n-pentane) to afford E-022b (14 mg, 42%) as an off-white solid. TLC (EtOAc/n-pentane, 1:1 v/v): R_{f}=0.40. ¹H NMR (500 MHz, DMSO) δ 7.69 (d, J=8.7 Hz, 1H), 7.54 (d, J=2.1 Hz, 1H), 7.41 (dd, J=8.7, 2.1 Hz, 1H), 6.23 (d, J=1.4 Hz, 1H), 6.12-6.03 (m, 1H), 5.67 (ddd, J=16.6, 9.7, 1.3 Hz, 1H), 5.02 (td, J=9.9, 5.1 Hz, 1H), 4.01 (q, J=7.0 Hz, 2H), 2.83 (dt, J=14.4, 9.3 Hz, 1H), 2.38 (d, J=1.2 Hz, 3H), 2.32-2.24 (m, 2H), 2.20 (dt, J=12.6, 6.2 Hz, 1H), 2.08-2.00 (m, 1H), 1.56-1.47 (m, 2H), 1.17 (t, J=7.1 Hz, 3H), 1.14-1.07 (m, 1H), 0.94 (q, J=12.1 Hz, 1H). ¹³C NMR (126 MHz, DMSO) δ 173.5, 160.0, 153.8, 153.2, 152.7, 142.8, 135.6, 131.8, 114.3, 114.2, 111.8, 104.4, 77.8, 59.9, 37.1, 34.6, 28.5, 28.0, 17.9, 14.1. HRMS (m/z): [M+Na]+ calcd. for C₂₃H₂₅N₁O₆Na: 434.1579, found 434.1578.

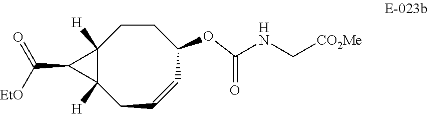

### COOEt-Exo-Equatorial-TCO-Glycine-OMe (E-023b)

To a H stirred solution of DPFPC (356 mg, 904 μmol) and DMAP (276 mg, 2.26 mmol) in dry DMF (1 mL) was added a solution of ethyl(1R,5S,8S,9R, E)-5-hydroxybicyclo[6.1.0]non-3-ene-9-carboxylate (E-02b, 95 mg, 452 μmol) and DIPEA (393 uL, 2.26 mmol) in DMF (1 mL). After the addition was complete, the mixture was covered from light. After overnight stirring, half of the reaction (1 mL) was transferred into a new, flame dried flask. Glycine methyl ester hydrochloride (30.2 mg, 339 μmol) was added to this solution. After 2.5 h, 2 additional equivalents of glycine methyl ester hydrochloride and 2.5 additional equivalents of DIPEA were added. When the reaction was complete, the reaction mixture was diluted with DCM and washed with 5% NaHCO₃ (5 mL), aqueous sat. NH₄Cl (5 ml) and brine (5 mL). The organic layer was dried with MgSO₄ and concentrated in vacuo and purified with silica gel column chromatography (30% EtOAc in heptane) to afford E-023b (20.2 mg, 42%). TLC (EtOAc/n-heptane, 1:1 v/v) R_{f}=0.33. ¹H NMR (500 MHz, CDCl₃) δ 5.91 (ddd, J=16.0, 9.2, 6.5 Hz, 1H), 5.67 (ddd, J=16.5, 9.6, 1.5 Hz, 1H), 5.15 (s, 1H), 4.95 (td, J=9.3, 8.7, 4.9, 1H), 4.09 (q, J=7.1, 2H), 3.97 (dd, J=5.6, 2.9, 2H), 3.76 (s, 3H), 2.91-2.81 (m, 1H), 2.40-2.28 (m, 2H), 2.22 (dt, J=12.8, 6.3, 1H), 2.16 (q, J=7.7, 1H), 1.55-1.45 (m, 1H), 1.36 (t, J=5.6, 1H), 1.26 (t, J=7.1, 3H), 1.22-1.17 (m, 1H), 0.82-0.72 (m, 1H). ¹³C NMR (126 MHz, CDCl₃) δ 174.5, 170.6, 156.0, 136.5, 131.1, 81.1, 60.6, 52.4, 42.6, 37.8, 35.0, 30.3, 29.2, 28.6, 26.5, 14.4. HRMS (m/z): [M+Na]+ calcd. for C₁₆H₂₃NO₆Na: 348.1423, found 348.1417.

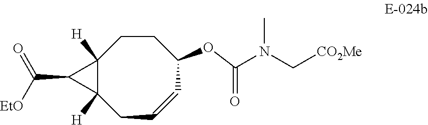

### COOEt-Exo-Equatorial-TCO-Sarcosine-OMe (E-024b

To a stirred solution of ethyl(1R,5S,8S,9R, E)-5-hydroxybicyclo[6.1.0]non-3-ene-9-carboxylate (E-02b, 37 mg, 177 μmol) in dry acetonitrile (1.5 mL) at 0° C., was added DPFPC (140 mg, 355 μmol), DIPEA (77.2 μL, 443 μmol), and DMAP (2.17 mg, 18 μmol). After the addition was complete, the mixture was shielded from light and allowed to warm up to ambient temperature overnight. After completion, the reaction mixture was diluted with diethyl ether (5 mL) and washed with water (2×5 mL). The organic layer was dried with MgSO₄ and concentrated in vacuo. Without further purification, the product was dissolved in dry acetonitrile (2.5 mL) and to this a solution of sarcosine methyl ester hydrochloride (49.5 mg, 355 μmol) and DIPEA (77.2 μL, 444 μmol) in dry acetonitrile (2.5 mL) was added. The reaction was shielded from light. Upon completion, the reaction mixture was diluted with EtOAc (10 mL) and washed with water (2×5 mL). The organic layer was dried with MgSO₄, concentrated in vacuo and purified with silica gel column chromatography (2%→5% acetone in toluene) yielding E-024b (15.1 mg, 25%) as an oil. TLC (acetone/toluene, 1:9 v/v) R_{f}=0.34. ¹H NMR (500 MHz, CDCl₃) rotamer 1: δ 5.96-5.86 (m, 1H), 5.72 (ddd, J=16.5, 9.6, 1.4, 1H), 4.96 (td, J=9.8, 5.3, 1H), 4.09 (q, J=7.3, 2H), 4.05-3.91 (m, 2H), 3.75 (s, 3H), 2.97 (s, 3H), 2.86 (dq, J=17.8, 9.1, 1H), 2.42-2.29 (m, 2H), 2.28-2.20 (m, 1H), 2.20-2.11 (m, 1H), 1.58-1.38 (m, 1H), 1.38-1.32 (m, 1H), 1.24 (t, J=7.2, 3H), 1.22-1.12 (m, 1H), 0.85-0.72 (m, 1H), rotamer 2: δ 5.96-5.86 (m, 1H), 5.63 (ddd, J=16.5, 9.7, 1.4, 1H), 4.96 (td, J=9.8, 5.3, 1H), 4.09 (q, J=7.3, 2H), 4.05-3.91 (m, 2H), 3.73 (s, 3H), 2.98 (s, 3H), 2.86 (dq, J=17.8, 9.1, 1H), 2.42-2.29 (m, 2H), 2.28-2.20 (m, 1H), 2.20-2.11 (m, 1H), 1.58-1.38 (m, 1H), 1.38-1.32 (m, 1H), 1.24 (t, J=7.2, 3H), 1.22-1.12 (m, 1H), 0.85-0.72 (m, 1H). ¹³C NMR (126 MHz, CDCl₃) rotamer 1: δ 174.5, 170.2, 155.8, 136.8, 131.6, 78.8, 60.6, 52.2, 50.6, 37.8, 35.3, 35.0, 30.3 29.3, 29.2, 28.6, 14.4, rotamer 2: δ 174.5, 170.2, 156.6, 136.6, 131.6, 79.0, 60.6, 52.2, 50.6, 37.8, 35.9, 35.0, 30.3 29.3, 29.2, 28.6, 14.4. HRMS (m/z): [M+Na]+ calcd. for C₁₇H₂₅NO₆Na: 362.1579, found 362.1565.

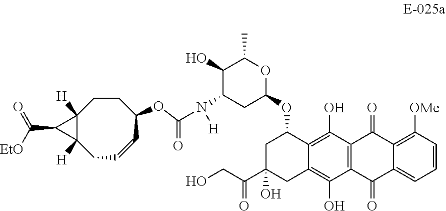

### COOEt-Exo-Axial-TCO-PNP (E-025a

COOEt-R-TCO-OH (E-02a, 99 mg, 471 μmol) was dissolved in dry DCM (10 mL) and pyridine (95 μL, 1.18 mmol) was added. A solution of 4-nitrophenyl chloroformate (85 mg, 424 μmol) in dry DCM (3 mL) was added. The reaction mixture was stirred for 4 h. before it was quenched with aqueous sat. NH₄Cl (10 ml). The phases were separated and the aqueous phase was extracted with DCM (2×15 mL) and the combined organic layers were dried with MgSO₄, concentrated in vacuo and purified with silica gel column chromatography (0%→10% EtOAc in n-pentane) to afford the Intermediate carbonate (65 mg, 70%) as an inseparable mixture of the product and starting 4-nitrophenyl chloroformate. TLC (EtOAc/n-pentane, 1:9 v/v): R_{f}=0.27. ¹H NMR (400 MHz, CDCl₃) δ 8.30-8.24 (m, 2H), 7.41-7.35 (m, 2H), 6.27 (ddd, J=16.9, 10.9, 5.9 Hz, 1H), 5.59 (dd, J=17.0, 3.3 Hz, 1H), 5.18 (q, J=3.0 Hz, 1H), 4.12 (qd, J=7.2, 1.6 Hz, 2H), 2.74 (dt, J=12.7, 6.2 Hz, 1H), 2.56-2.46 (m, 1H), 2.33-2.19 (m, 2H), 2.09-2.00 (m, 1H), 1.88 (t, J=13.8 Hz, 1H), 1.63-1.55 (m, 2H), 1.53-1.47 (m, 1H), 1.27 (t, J=7.1 Hz, 3H). ¹³C NMR (101 MHz, CDCl₃) δ 174.9, 155.6, 150.1, 146.0, 133.3, 128.2, 125.4, 121.8, 75.9, 60.8, 37.5, 31.4, 29.5, 26.6, 20.7, 14.4.

HRMS (m/z): [M+Na]+ calcd. for C₁₉H₂₁N₁O₇Na: 398.1215, found 398.1228. The Intermediate carbonate (6.5 mg, 17 μmol) was dissolved in dry DMF (2 mL) and triethylamine (2.6 μL, 19 μmol) and Doxorubicin hydrochloride (9.0 mg, 16 μmol) were added. The reaction mixture was stirred in the dark overnight. LCMS indicated still a significant amount of starting material so the temperature was raised to 40° C. and the reaction was stirred for another night. The reaction mixture was concentrated in vacuo and the crude product was purified using reversed-phase preparative HPLC (0→100% MeCN (0.1% formic acid) in MiliQ (0.1% formic acid)) and lyophilized to afford E-025a (3.8 mg, 30%). TLC (DCM/MeOH, 9:1 v/v): R_{f}=0.07. ¹H NMR (500 MHz, CDCl₃) δ 14.02 (s, 1H), 13.29 (s, 1H), 8.07 (d, J=7.7 Hz, 1H), 7.81 (td, J=8.1, 2.1 Hz, 1H), 7.44-7.39 (m, 1H), 6.16-5.99 (m, 1H), 5.58-5.48 (m, 2H), 5.37-5.30 (m, 1H), 5.18-5.11 (m, 1H), 5.03-4.97 (m, 1H), 4.61-4.49 (m, 1H), 4.21-4.14 (m, 2H), 4.11 (s, 3H), 4.09-4.03 (m, 1H), 3.92-3.83 (m, 1H), 3.73-3.66 (m, 1H), 3.35-3.25 (m, 1H), 3.11-3.03 (m, 1H), 2.47-2.32 (m, 2H), 2.09-2.02 (m, 2H), 1.93-1.86 (m, 2H), 1.73 (d, 7H), 1.28-1.26 (m, 6H). ¹³C NMR (126 MHz, CDCl₃) δ 214.0, 187.3, 186.9, 175.1, 161.2, 156.3, 155.8, 154.9, 135.9, 135.7, 133.7, 131.5, 129.9, 129.7, 121.0, 120.0, 118.6, 111.7, 111.6, 100.8, 71.4, 69.7, 67.4, 65.6, 60.6, 56.8, 50.9, 47.0, 46.0, 37.5, 35.8, 34.9, 32.0, 31.0, 29.8, 26.9, 22.8, 21.0, 17.0, 14.2. HRMS (m/z): [M+Na]+ calcd. for C₄₀H₄₅N₁O₁₅Na: 802.2686, found 802.2710.

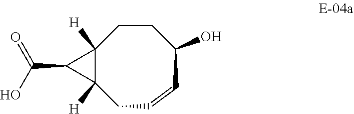

### Synthesis of (1S,5R,8R,9S, E)-5-hydroxybicyclo[6.1.0]non-3-ene-9-carboxylic acid (E-04a

To a stirred solution of E-02a (500 mg, 1 equiv, 2.40 mmol) in THF (8 mL) and water (4 ml) was added LiOH (172 mg, 3.0 equiv, 7.20 mmol). The mixture was stirred at 50° C. and covered with aluminum foil. After completion was seen on TLC, EtOAc (5 ml) and 1M aqueous HCl (5 ml) were added to the mixture. The organic layer was washed with brine (5 ml) and both aqueous layers were back-extracted with EtOAc (2×5 ml). The combined organic layers were dried over MgSO₄ and concentrated to afford the crude product in 376 mg (87% of theory) yield. ¹H NMR (400 MHz, D₂O) δ 6.18 (ddd, J=16.9, 10.8, 5.8 Hz, 1H), 5.63 (dd, J=17.1, 3.4 Hz, 1H), 4.35 (q, J=3.0 Hz, 1H), 2.66 (dt, J=12.8, 6.4 Hz, 1H), 2.43 (t, J=11.8 Hz, 1H), 2.14 (dd, J=15.3, 11.4 Hz, 1H), 1.90-1.74 (m, 3H), 1.44 (t, J=5.9 Hz, 1H), 1.38-1.30 (m, 1H), 1.28-1.21 (m, 1H). ¹³C NMR (101 MHz, D₂O) δ 186.83, 134.80, 133.14, 70.48, 41.13, 32.10, 31.07, 27.68, 25.40, 21.35. RF (methanol/dichloromethane 1-9) 0.23.

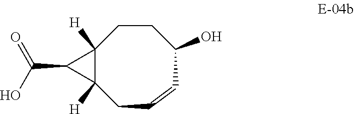

### Synthesis of (1S,5R,8R,9S, E)-5-hydroxybicyclo[6.1.0]non-3-ene-9-carboxylic acid (E-04b

To a stirred solution of E-02b (500 mg, 1 equiv, 2.40 mmol) in THF (8 mL) and water (4 ml was added LiOH (172 mg, 3.0 equiv, 7.20 mmol). The mixture was stirred at 50° C. and covered with aluminum foil. After completion was seen on TLC, EtOAc (5 ml) and 1M aqueous HCl (5 ml) were added to the mixture. The organic layer was washed with brine (5 ml) and both aqueous layers were back-extracted with EtOAc (2×5 ml). The combined organic layers were dried over MgSO₄ and concentrated to afford the crude product in 370 mg (85% of theory) yield. ¹H NMR (400 MHz, D₂O) δ 5.93 (ddd, J=15.9, 9.0, 6.4 Hz, 1H), 5.63 (ddd, J=16.5, 9.5, 1.4 Hz, 1H), 4.09 (td, J=9.7, 5.3 Hz, 1H), 2.86-2.69 (m, 1H), 2.30-2.17 (m, 2H), 2.06 (ddd, J=12.8, 6.9, 5.4 Hz, 1H), 2.00-1.86 (m, 1H), 1.39 (dt, J=12.9, 10.6 Hz, 1H), 1.18 (t, J=5.7 Hz, 1H), 0.95 (ddt, J=11.3, 8.9, 4.9 Hz, 1H), 0.70 (dt, J=15.5, 11.2 Hz, 1H). ¹³C NMR (101 MHz, D₂O) δ 184.19, 138.61, 131.78, 75.51, 37.57, 36.76, 31.87, 29.93, 28.18, 28.14. RF (methanol/dichloromethane 1:9) 0.20.

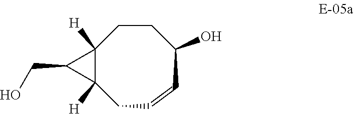

### Synthesis of (1R,4R,8S,9S, E)-9-(hydroxymethyl)bicyclo[6.1.0]non-5-en-4-ol (E-05a

To a suspension of lithium aluminum hydride (14.2 mg, 2.4 equiv, 0.38 mmol) in dry diethyl ether (750 μl), was added a solution of E-02a (40 mg, 2 equiv, 0.16 mmol) in dry diethyl ether (750 μl) at 0° C. The mixture was stirred at ambient temperature for one hour. After completion was seen on L, an aqueous solution of 1.0 M HCl (800 μl) was dropwise added to reaction mixture. Extra diethyl ether (5 ml) and aqueous 1.0 M HCl solution (5 ml) were added to the reaction mixture. The organic layer was collected and washed with brine (5 ml). The aqueous layers were back extracted with diethyl ether (2×5 ml). The combined organic layers were dried over MgSO₄ and concentrated to afford the desired product in 26 mg yield. The crude was purified over silica (8 g) with 10% MeOH in DCM to afford a clear oil in 20 mg (62% of theory) yield. ¹H NMR (400 MHz, CDCl₃) δ 6.27 (ddd, J=16.7, 10.8, 5.9 Hz, 1H), 5.54 (dd, J=16.8, 3.2 Hz, 1H), 4.35 (q, J=3.1 Hz, 1H), 3.52-3.43 (m, 2H), 2.65 (dt, J=12.8, 6.5 Hz, 1H), 2.40 (t, J=11.6 Hz, 1H), 2.25 (dd, J=16.3, 11.8 Hz, 1H), 1.99-1.88 (m, 1H), 1.88-1.76 (m, 1H), 1.77-1.66 (m, 1H), 1.04 (p, J=6.4 Hz, 1H), 0.81 (dd, J=14.7, 7.1 Hz, 1H), 0.65 (td, J=8.8, 6.2 Hz, 1H). ¹³C (NMR 101 MHz, CDCl₃) δ 132.92, 131.28, 68.68, 67.72, 39.90, 29.70, 25.98, 22.41, 22.40, 19.88. RF (methanol/dichloromethane 1:9) 0.22.

### Example 3—Stability Test

An NMR tube was charged with a solution of E-04a (41 mg) in deuterated PBS buffer (500 μl). The NMR sample was placed in a water bath at 37° C. while being covered from light by aluminum foil. The sample showed no changes in the ¹H-NMR spectrum after 7 days.

### Example 4—Kinetics Study of Click Reaction

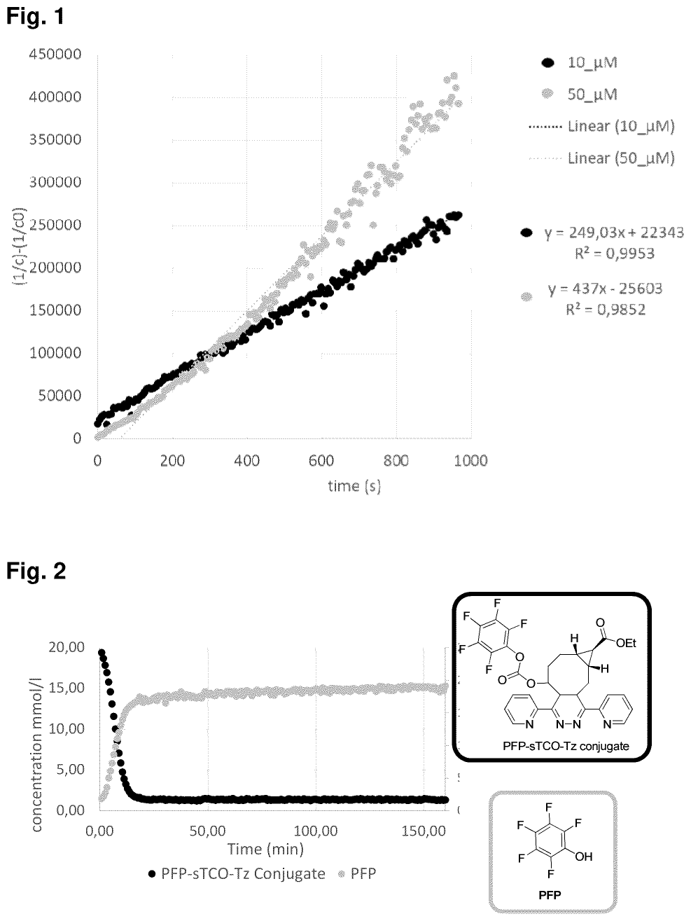

The reaction rate in a TCO-Tz click reaction of E-02a was measured under similar conditions as described in Versteegen et al., Angewandte Chemie International Edition 2013, 52 (52), 14112-14116. The second order reaction constant of the reaction between 3,6-di(pyridin-2-yl)-1,2,4,5-tetrazine and E-02a was determined under second order conditions in MeCN at 20° C. by UV spectroscopy. A cuvette was filled with MeCN (2.8 mL) and equilibrated at 20° C. A stock solution of tetrazine in DMSO (100 μl) was added to the cuvette. The absorption of the tetrazine moiety was measured at 540 nm. Next, the cuvette was removed from the apparatus to add a stock solution of E-02a in DMSO (100 μl) and briefly mixed the solution by shaking the cuvette before placing it back into the apparatus. The absorption at 540 nm was measured for 15 minutes. From this absorption at 540 nm, the concentration of tetrazine was calculated using a molar absorption coefficient of ε=430 M⁻¹ cm⁻¹. The second order rate constant k2 was obtained from the slope of a plot of (1/c−1/c0) versus time. The calculated reaction rates at a concentration of 10 μM and 50 μM were respectively 249 M⁻¹s⁻¹ and 437 M⁻¹s⁻¹. FIG. 1 shows that the kinetics of TCO E-02a and 3,6-di(pyridin-2-yl)-1,2,4,5-tetrazine with a concentration of 10 and 50 μM at 20° C. in MeCN showed rates of 249 M⁻¹s⁻¹ and 437 M⁻¹s⁻¹ respectively. TCO reactions known from the prior art (Versteegen et al., Angewandte Chemie International Edition 2013, 52 (52), 14112-14116, DOI: 10.1002/anie.201305969) were assessed at 83.3 μM and afforded a lower rate of 62, 5 M⁻¹s⁻¹ under similar conditions.

### Example 5—Kinetics Study of Release Reaction

The ¹⁹F NMR (377 MHz) spectrum of an NMR-tube filled with a solution of E-03a (5 mg, 1.0 equiv, 11.9 μmol) in CDCl₃ (500 μl) was measured. Next, a solution of 3,6-di(pyridin-2-yl)-1,2,4,5-tetrazine (3.1 mg, 1.1 equiv, 13 μmol) in CDCl₃ (100 μl) was added to the NMR tube. The tube was vigorously shaken and placed back in the NMR apparatus. The sample was measured within two minutes after the addition of the second compound. Every 48 seconds a measurement (number of scans: 4, relaxation delay: 5.8 see, range: −147.0 to −178.5 ppm) was taken of the sample. After 160 minutes the measurements were stopped. Almost complete release of PFP was seen after 20 minutes. FIG. 2 shows the results of a ¹⁹F-NMR kinetic study of the click-to-release reaction of 20 mM TCO (E-03a) with 3,6-di(pyridin-2-yl)-1,2,4,5-tetrazine at 20° C. Complete release of pentafluorophenolate was observed after 20 minutes upon the click reaction with dipyridyltetrazine. When using E-03b instead, release was found to be several factors slower.

Further investigation of the E-03a click-to-release reaction at lower temperatures showed that the release was instantaneous upon tautomerization from the click product. ¹⁹F-NMR kinetic study of the click reaction of 40 mM with dipyridyl tetrazine was performed at controlled temperatures. At −20° C. the click conjugate of the compound of the invention and dipyridyl tetrazine could be observed and did not show any formation of pentafluorophenol. Only after elevating the temperature to +10° C., the pentafluorophenyl carbonate was released from the conjugated product.
Release of payload from compounds of the invention was not only found to be fast, but also complete. Versteegen et al. (Angewandte Chemie International Edition 2018, 57 (33), 10494-10499 DOI: 10.1002/ange.201800402) tested various payloads on the allylic position of TCO compounds known from the prior art and observed only a maximum of 60% release under conditions that were comparable to those used in our ¹⁹F-NMR studies. The fastest payload release observed with their compounds afforded less than 20% release over 250 minutes. Compounds of the present invention showed near complete release within 20 minutes.

### Example 6—Kinetics Study of Fluorophore Release

A procedure adapted from prior art was followed (Fan, X. et al., Optimized tetrazine derivatives for rapid bioorthogonal decaging in living cells. Angew. Chem. 2016, 128 (45), 14252-14256.)

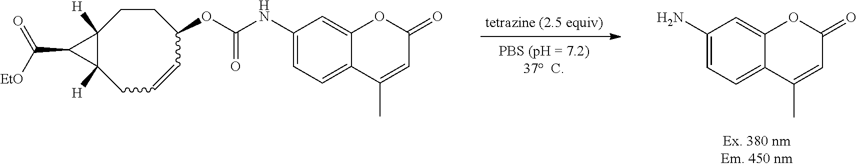 [SMILES: CCOC(=O)[C@H]1[C@@H]2CC[C@@H](OC(=O)Nc3ccc4c(C)cc(=O)oc4c3)C=CC[C@@H]21 ; Cc1cc(=O)oc2cc(N)ccc12]

Stock solutions (all in DMSO)
Stock A (R): E-022a (10 mmolar)
Stock A (NR): E-022b (10 mmolar)
Stock B (DM): 3,6-Dimethyl-1,2,4,5-tetrazine (DM) (20 mmolar)
Stock B (BP): 3,6-Di-2-pyridyl-1,2,4,5-tetrazine (BP) (20 mmolar)
Stock PC: 7-Amino-4-methylcoumarin (10 mmolar)
   2.5 μL of the tetrazine stock (DM or BP) was mixed with 395.5 μL of PBS (pH=7.2). 2 μL of the TCO stock (A (R) or A (NR)) was added to this. The reaction mixtures were incubated at 37° C. under mixing (300 rpm). Subsequently, the fluorescence (F1) was measured (ex: 380 nm, em. 450 nm) at certain time points after the addition of the TCO by aliquoting 30 μL in a 384 wells plate (Greiner, black non-binding flat bottom) and directly measuring with a Tecan Spark plate-reader. As control experiment the maximum amount of released AMC was measured (F2, Stock PC without tetrazine or TCO). Negative control experiments revealed no significant fluorescent signal for either exclusively the tetrazine or the TCO's. The release efficiency (%) was calculated: F1/F2×100%. All experiments were executed in triplo and the data was processed using GraphPad Prism (version 9.0). The Kₑₗᵢₘ. values were determined by first-order exponential one phase decay approximations. E-022a with DM displayed a Kₑₗᵢₘ. value of 8.15*10⁻⁵S⁻¹. For BP this value was 2.96*10⁻⁶S⁻¹. As for example 5, release was found to be slower for the E-022b analogue.

### Example 7—Kinetics Study of Doxorubicin Release

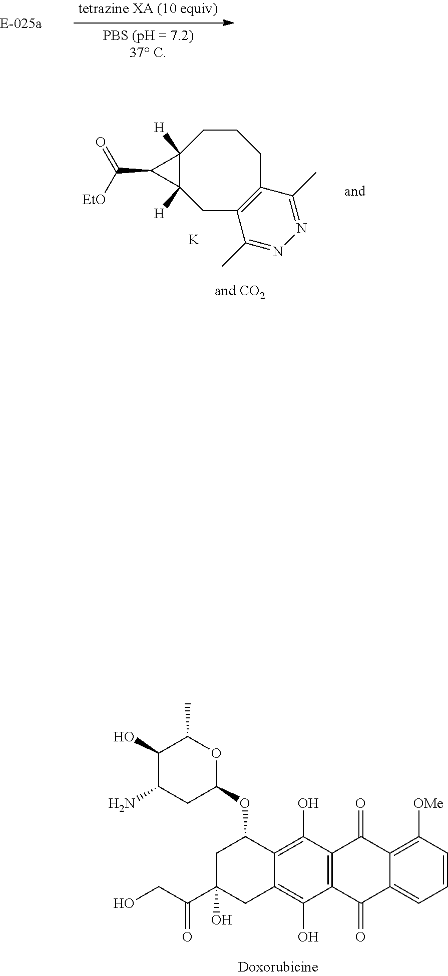

Stock solutions (all in DMSO)
Stock E-025a: E-025a (10 mmolar)
Stock Ttz: 3,6-dimethyl-1,2,4,5-tetrazine (XA, 20 mmolar)
   Procedure: 100 μL of the TCO-Dox stock was added to 900 μL of PBS (pH=7.2) and HPLC analysis was done which only indicated starting material (t=13.08 min). Hereafter the reaction was incubated at 37° C. under mixing (300 rpm) and 500 μL of the Ttz stock was added. At certain time points after the addition of the Ttz the reaction mixture was analyzed by aliquoting 200 μL and analysis by HPLC/LCMS. Directly after the addition of the tetrazine (t=6.11 min) almost instant release of doxorubicin (t=7.57 min) and formation of the elimination product (t=7.19 min) was observed.

1. A compound of general formula (I), or a salt thereof: 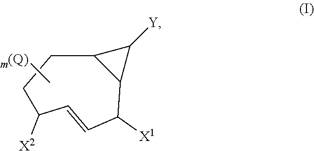
wherein
one of X¹ and X² is —R^{a} or —Z¹—C(═Z²)—R^{b}, and the other is —H;
Y is chosen from —C(═Z³)—R^{c} or —CH₂—R^{a};
Z¹, Z², and Z³ are in each instance chosen independently from O, S or NQ^{N};
R^{a}, R^{b}, R^{c}, and R^{d} are in each instance chosen independently from —O—Pr, —S—Pr, —NQ^{N1}Q^{N2}, a leaving group, a pharmaceutically active substance, or a targeting moiety;
Pr is in each instance chosen independently from —H, a linear, branched, or cyclic C1-6 alkyl or acyl, or a protecting group, wherein acyl or alkyl are optionally unsaturated and optionally substituted with halogen, alkoxy, —C(═O)O(CH₂)₀₋₄H, or haloalkoxy;
Q, Q^{N}, Q^{N1}, and Q^{N2} are in each instance chosen independently from —H or a linear, branched or cyclic C1-6 alkyl, wherein alkyl is optionally unsaturated and optionally substituted with halogen, alkoxy, or haloalkoxy; and
m is 0, 1, 2, 3 or 4.

2. The compound according to claim 1, wherein m is 0.

3. The compound according to claim 1, wherein X² is —R^{a} or —O—C(═O)—R^{b}, and X¹ is —H.

4. The compound according to claim 1, wherein Y is —C(═O)—R^{c} or —CH₂—R^{d}.

5. The compound according to claim 1, wherein the protecting group is acetyl (Ac), benzoyl (Bz), benzyl (Bn), β-methoxyethoxymethyl (MEM), dimethoxytrityl, bis-(4-methoxyphenyl)phenylmethyl (DMT), methoxymethyl (MOM), methoxytrityl (MMT), p-methoxybenzyl (PMB), p-methoxyphenyl (PMP), methylthiomethyl, pivaloyl (Piv), tetrahydropyranyl (THP), tetrahydrofuranyl (THF), trityl (Tr), silyls such as trimethylsilyl (TMS), tert-butyldimethylsilyl (TBDMS), tri-iso-propylsilyloxymethyl (TOM), and triisopropylsilyl (TIPS), or ethoxyethyl (EE).

6. The compound according to claim 1, wherein the leaving group is a halogen, or wherein the leaving group is

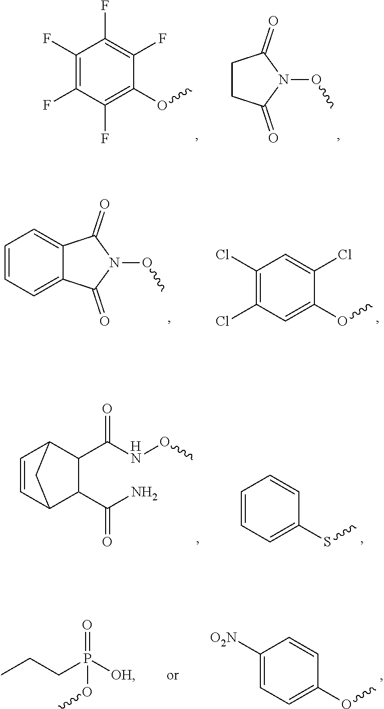

7. The compound according to claim 1, wherein the compound is of general formula (V-OO), (V-HO), (V-OH) or (V-HH):

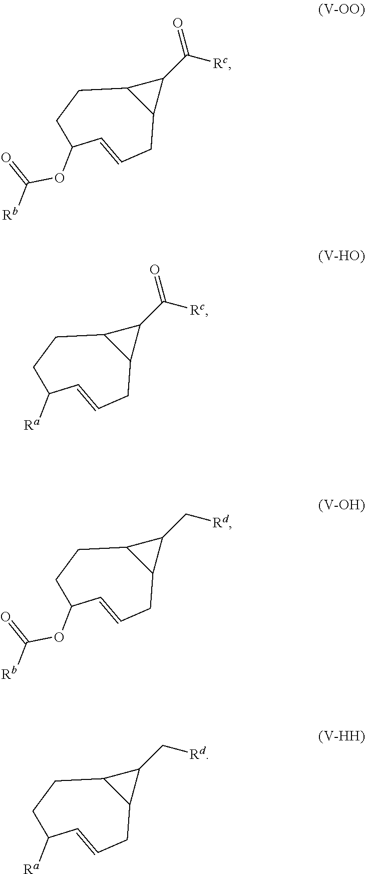

8. The compound according to claim 1, wherein the compound is of general formula (III-a), (III-c), or (III-e):

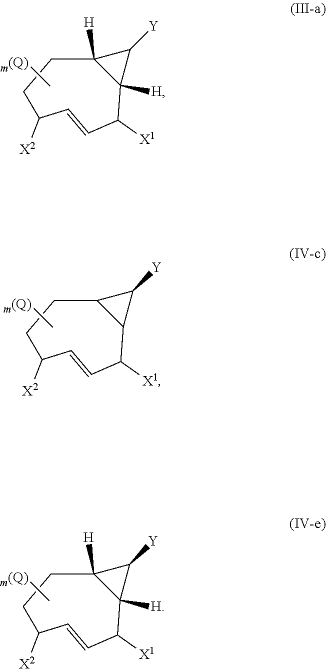

9. The compound according to claim 1, wherein the compound is of general formula (II-a) or (VI-a) or an enantiomer thereof:

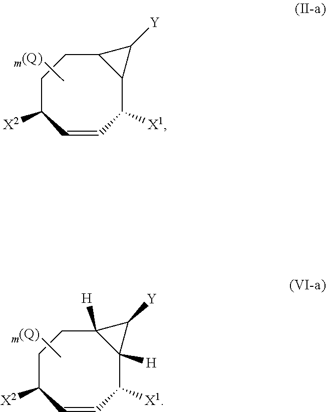

10. The compound according to claim 1, wherein the compound is of general formula (VII-O) or (VII-OH) or an enantiomer thereof, wherein R^{b}, R^{C} and R^{d} are in each instance chosen independently from —OH or a leaving group:

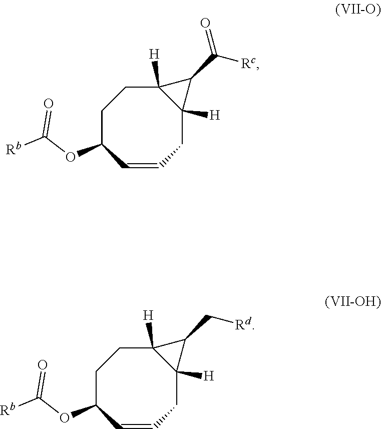

11. (canceled)

12. A method for producing a conjugate, the method comprising the steps of:
i) providing a compound as defined in claim 1;
ii) providing a second compound;
iii) reacting the compound provided in step i) with the second compound to form a conjugate; and optionally
iv) isolating the conjugate obtained in step iii).

13. A composition comprising a compound as defined in claim 1, and a pharmaceutically acceptable excipient.

14.-15. (canceled)

16. The method according to claim 12, wherein the second compound is a protein.

17. The method according to claim 12, wherein the second compound is an antibody.

18. The composition according to claim 13, wherein the composition comprises a tetrazine.

19. The composition according to claim 16, wherein the composition comprises a dipyridyl tetrazine.
